(19) United States
(12) Patent Application Publication
(10) Pub. No.: US 2023/0260665 A1
(43) Pub. Date: Aug. 17, 2023

(54) MODELING FOR COMPLEX OUTCOMES USING SIMILARITY AND MACHINE LEARNING ALGORITHMS

(71) Applicant: The Ronin Project, Inc., San Mateo, CA (US)

(72) Inventors: Christine SWISHER, San Diego, CA (US); Stephen WEAVER, Cottonwood Heights, UT (US); Renee GEORGE, San Diego, CA (US); Andrew WEST, Chicago, CA (US); Alex GRAFF, San Mateo, CA (US); Neehar MUKNE, San Mateo, CA (US)

(21) Appl. No.: 18/078,894

(22) Filed: Dec. 9, 2022

Related U.S. Application Data
(60) Provisional application No. 63/287,941, filed on Dec. 9, 2021.

(51) Int. Cl. G16H 50/70; G16H 15/00; G16H 10/60; G06F 18/2413

(52) U.S. Cl. CPC G16H 50/70; G16H 15/00; G16H 10/60; G06F 18/2413

(57) ABSTRACT

Described herein are systems and methods for modeling complex outcomes using similarity and machine learning algorithms. Machine learning algorithms and models can be implemented on platforms comprising one or more user interfaces and an insight engine. In these embodiments, insight engine comprises a machine learning software algorithm (or module) configured to ingest data and generate insights.

### CROSS REFERENCE

This application claims the benefit of U.S. Provisional Application No. 63/287,941, filed Dec. 9, 2021, which application is incorporated herein by reference.

### BACKGROUND

Given, in part at least, the breadth of medical information now widely available along with the rapid rate at which new medical information is discovered (through, e.g., new studies) as well as the rate of development of new therapies, providing effective healthcare to patients has become difficult for today's healthcare providers. Current methods for modeling this information fail to effectively process and evaluate large and complex data sets for various diseases.

Cancer, in particular, has long been and remains a disease that is difficult to treat given, among other things, the complexity of the disease process, the differences between cancer types, and the differences between patients. In addition, while promising therapeutics and therapeutic regimens are being constantly formulated and developed, delivering the right therapeutic and/or therapeutic regimen to a patient is a complicated process for the health care provider.

### SUMMARY

Described herein are systems and methods utilizing machine learning algorithms for enhanced data processing and analysis. In some embodiments, machine learning algorithms and models process medical data such as electronic medical records, laboratory results, medical imaging, and other relevant medical information to identify clusters of data points based on multi-dimensional parameters. These clusters can form the basis for multi-outcome based predictive models which can be integrated into a data insights engine to generate analytic insights to inform the decision-making process. Single-outcome predictive models can also be generated and used to generate additional analytic insights.

The proliferation of molecular approaches for analyzing cancers has yielded greater insight into the complexity and variation of cancers. Currently, more than 200 types of cancer have been identified, each of which can be further categorized into various stages and subtypes. For example, 7 lung cancer subtypes were discovered in 2016 alone. The year 2017 was estimated to have had over 1.6 million new cancer diagnoses and around 600 thousand cancer deaths. However, determining the appropriate course of action for a patient can be challenging due to the unique heterogeneity of the particular cancer as well as the circumstances of the individual. Moreover, the number of opportunities in a patient's journey where machine learning could provide clinical decision support is numerous and can number in the hundreds.

The concept of using artificial intelligence to make predictions about specific cancers relating to diagnosis and treatment may seem like a useful application of this technology, but the number of different cancers and subtypes and stages can make this approach impractical to cover the majority of the needed use cases. Indeed, the heterogeneous nature of cancers diminishes the predictive power of cancer-specific machine learning models because such models are only as effective as the training dataset. Thus, once the overall data set is categorized according various cancer types, subtypes, grades, and other potentially relevant information such as molecular biomarkers, only a small amount of data may be available for a given feature combination corresponding to a particular cancer. For example, relevant information can include age of the subject that allows reference data states to be categorized or filtered as pediatric or geriatric data. The result is the need for a proliferation of machine learning models with a bespoke model tailored to each kind of cancer, which is a solution that is unfeasible from a practical standpoint due to the paucity of relevant training data at that degree of resolution. In other words, simply applying machine learning generally to the cancer problem faces significant hurdles that hinder the need to guide individual patients through their unique journeys. Accordingly, disclosed herein are platforms, systems, devices, media, and methods that provide a unique solution to this technical problem.

In some aspects, disclosed herein are platforms, systems, devices, media, and methods for providing analytical insights that guide patients and/or their healthcare providers in their personalized journey of care. In some cases, a patient or healthcare provider can enter an input such as a query or free text question (e.g., unstructured search query). Analytical insights may be provided in response to the user input, for example, including summary statistics for a grouping of most similar reference patient/subject states. Although the technical solution provided herein is particularly well-suited for addressing cancer, it is also useful for other diseases, especially complex diseases that are not homogeneous in nature across the patient population. For example, interstitial lung disease is a broad disease category that lacks targeted treatment, has poor outcomes, and is characterized by a broad range of onset and aggression that make medical decision-making difficult. Mental health conditions such as addiction or substance abuse are also known to have wildly varying treatment outcomes due to disparities in patient and socioeconomic attributes. The consequences for failure can be high, and there is a lack of a platform that facilitates personalized treatment to improve outcomes. In some cases, similar cases are identified for a particular patient without requiring the use of a machine learning model configured for the particular cancer type/subtype/molecular features. Instead, disclosed herein is an innovative approach that allows for the identification of similar cases to that of the patient which can be used to provide clinical insights without having to parse out the cases or dataset according to specific cancers, subtypes, etc. Alternatively or in combination, disclosed herein is an innovative approach in finding similar cases to that of a patient by using lower dimensional numerical representations created using Deep Learning techniques such as Variational Auto-Encoders. Such methods allow representing each patient as lower dimensional numeric representations (or vectors) in a continuous space. This approach enables patient similarity calculations to be performed on the fly (cosine distance) or for clustering patients into cohorts or groupings. In some embodiments, the patient information is extracted from one or more different data sources such as, for example, laboratory test results, medical records, doctor notes, patient questionnaires, and other relevant sources. Natural language processing and/or semantic search may be performed to extract features from the data sources. This feature extraction can be performed on all patients within the database. Next, the various patient cases can be aligned. While conventional approaches to alignment may simply temporally align cases according to a key event, for example, aligned according to diagnosis of cancer or determination of a particular grade, the instant disclosure provides a unique solution for generating a more relevant alignment. In some cases, the time course alignment is generated based on a comparison of patient cases according to temporal events in their medical histories.

One advantage of the present disclosure is that the analytical insights can be provided with transparency to promote trust by the healthcare provider or patient. In some implementations, the analytical insights are provided with or comprise the specific features that are important for identifying the appropriate grouping from which the insights are derived. For example, a physician reviewing the summary statistics for a patient grouping identified as most closely corresponding (e.g., highest statistical similarity) to their patient may notice certain discrepancies that make the comparison out of place. In this example, the physician may notice a large age difference between their patient and the grouping. Thus, providing the physician with the features that were used to identify this grouping allows them to draw inferences about the appropriateness of the grouping. Moreover, this information can provide additional insight as to medical decisions. For example, an important feature for determining the grouping may be the presence of hypertension in both the grouping and the patient. A physician may take notice of this information and consider putting their patient on statins. Thus, the transparency of the analytical insights provided herein can enable more informed medical decision-making that would be more difficult when simply providing black box machine learning predictions.

Described herein is a computer-implemented system for data state alignment and cohort generation, the system comprising: a processor; a non-transitory computer readable storage medium encoded with a computer program that causes the processor to: receive user input for querying a database comprising a plurality of reference data states based on one or more subject data states; align said one or more subject data states with said plurality of reference data states; identify one or more groups of similar reference data states based on aligning said one or more subject data states with said plurality of reference data states; and generate summary statistics comprising one or more analytical insights based on said one or more groups of similar reference data states. In some embodiments, said user input comprises a free-form question. In some embodiments, said processor is further caused to extract one or more intents from said user input. In some embodiments, said one or more intents define at least one target variable, exclusion criteria, a type of comparison, or any combination thereof. In some embodiments, said one or more intents correspond to a template. In some embodiments, said type of comparison comprises individualized comparison, a group comparison, or a ranking. In some embodiments, said user input is processed using a natural language processing algorithm to extract said one or more intents, one or more diseases, one or more health problems, or one or more treatments. In some embodiments, said natural language processing algorithm is configured to perform natural language understanding on said user input. In some embodiments, said user input is processed using an ontology map. In some embodiments, an elastic search is used to generate a data set of said plurality of reference data states allowing retrieval of hierarchical data. In some embodiments, said data set is structured as an index table of said plurality of reference data states that provide snapshots of a plurality of reference subjects. In some embodiments, said elastic search is used to generate said index table of said plurality of reference data states, wherein said one or more subject data states are aligned against one or more reference data states within said index table. In some embodiments, said index table is based on said one or more intents extracted from said user input. In some embodiments, said plurality of reference data states corresponds to multiple time points for a plurality of reference subjects. In some embodiments, each of said plurality of reference data states corresponds to a time point for a reference subject. In some embodiments, each of a subset of said plurality of reference subjects has two or more reference data states corresponding to two or more time points. In some embodiments, a reference subject comprises multiple reference data states corresponding to multiple time points, wherein a reference data state is selected from said multiple reference data states for alignment with said one or more subject data states. In some embodiments, the one or more groups of similar reference data states are identified using the most similar reference data state for each of said plurality of reference subjects. In some embodiments, said one or more subject data states comprises subject information. In some embodiments, said subject information comprises a plurality of features comprising one or more of: (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, (15) treatment goal, (16) socioeconomic factors, or (15) confounding factors. In some embodiments, said processor is further configured to parse said one or more subject data states into said one or more groups based on said subject information. In some embodiments, said processor is further configured to recommend and answer one or more questions determined to be associated with said user input. In some embodiments, said processor is further configured to provide a recommender module that identifies said one or more questions associated with said user input, optionally wherein said recommender system performs an item-user or item-item collaborative filtering to identify said one or more questions associated with said user input. In some embodiments, said one or more processors are further caused to output said summary statistics comprising said one or more analytical insights. In some embodiments, said one or more processors are further caused to generate and output a comparison of a group comprising said one or more subject data states to another group, population, or a reference material. In some embodiments, said comparison compares summary statistics for said group with said another group, said population, or said reference material. In some embodiments, said one or more groups of similar reference data states is determined to be most similar to said one or more subject data states. In some embodiments, said one or more processors are further caused to output feature importance for one or more features used to determine said one or more groups of similar reference data states as being most similar to said one or more subject data states. In some embodiments, said one or more processors are further caused to output a ranked list of reference data states selected from said plurality of reference data states most similar to said one or more subject data states. In some embodiments, the one or more groups are identified using a similarity algorithm configured to determine statistical similarity between said one or more subject data states and said plurality of reference data states. In some embodiments, said similarity algorithm is a non-machine learning algorithm. In some embodiments, said similarity algorithm is configured to calculate Euclidean distance. In some embodiments, said one or more subject data states comprises health data for one or more subjects retrieved from an electronic medical record. In some embodiments, said health data comprises a plurality of features extracted from said electronic medical record using a natural language processing algorithm. In some embodiments, said natural language processing algorithm comprises one or more rules for keyword identification, unit conversion, internal consistency, or any combination thereof. In some embodiments, said natural language processing algorithm comprises a natural language processing model configured to annotate said electronic medical record with gold standard labels. In some embodiments, said processor is further configured to provide a healthcare provider application comprising a healthcare provider interface configured to present a healthcare provider with said statistical summary comprising said one or more analytical insights. In some embodiments, said healthcare provider interface comprises a first plurality of portals, wherein at least one of said first plurality of portals comprises a patient context data grouping comprising a subset of said reference data states within said one or more groups. In some embodiments, at least one of said first plurality of portals comprises an outcomes navigator data grouping comprising outcome information for said subset of said reference data states within said one or more groups. In some embodiments, said outcome information comprises one or more of cancer survival, cancer progression, adverse event status, treatment status, or mortality. In some embodiments, said adverse event status comprises neutropenia, leucopenia, thrombocytopenia, fatigue, pain, mucositis, skin rash, nausea, vomiting, constipation, diarrhea, cognitive dysfunction, nerve damage, appetite loss, organ damage, or any combination thereof. In some embodiments, said patient context data grouping comprises a second plurality of portals comprising an interactive timeline of a disease of said one or more subjects, interactive radiology imaging of said one or more subjects, a medical history of said one or more subjects, a current status of said one or more subjects , or any combination thereof

Described herein is a computer-implemented method for data state alignment and cohort generation, comprising: receiving user input for querying a database comprising a plurality of reference data states based on one or more subject data states; aligning said one or more subject data states with said plurality of reference data states; identifying one or more groups of similar reference data states based on aligning said one or more subject data states with said plurality of reference data states; and generating summary statistics comprising one or more analytical insights based on said one or more groups of similar reference data states. In some embodiments, said user input comprises a free-form question. In some embodiments, the method further comprises extracting one or more intents from said user input. In some embodiments, said one or more intents define at least one target variable, exclusion criteria, a type of comparison, or any combination thereof. In some embodiments, said one or more intents correspond to a template. In some embodiments, said type of comparison comprises individualized comparison, a group comparison, or a ranking. In some embodiments, said user input is processed using a natural language processing algorithm to extract said one or more intents, one or more diseases, one or more health problems, or one or more treatments. In some embodiments, said natural language processing algorithm is configured to perform natural language understanding on said user input. In some embodiments, said user input is processed using an ontology map. In some embodiments, an elastic search is used to generate a data set of said plurality of reference data states allowing retrieval of hierarchical data. In some embodiments, said data set is structured as an index table of said plurality of reference data states that provide snapshots of a plurality of reference subjects. In some embodiments, said elastic search is used to generate said index table of said plurality of reference data states, wherein said one or more subject data states are aligned against one or more reference data states within said index table. In some embodiments, said index table is based on said one or more intents extracted from said user input. In some embodiments, said plurality of reference data states corresponds to multiple time points for a plurality of reference subjects. In some embodiments, each of said plurality of reference data states corresponds to a time point for a reference subject. In some embodiments, each of a subset of said plurality of reference subjects has two or more reference data states corresponding to two or more time points. In some embodiments, a reference subject comprises multiple reference data states corresponding to multiple time points, wherein a reference data state is selected from said multiple reference data states for alignment with said one or more subject data states. In some embodiments, the one or more groups of similar reference data states are identified using the most similar reference data state for each of said plurality of reference subjects. In some embodiments, said one or more subject data states comprises subject information. In some embodiments, said subject information comprises a plurality of features comprising one or more of: (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, or (15) treatment goal. In some embodiments, the method further comprises parsing said one or more subject data states into said one or more groups based on said subject information. In some embodiments, the method further comprises recommending one or more questions determined to be associated with said user input and answering said one or more questions. In some embodiments, item-user or item-item collaborative filtering is performed to identify said one or more questions associated with said user input. In some embodiments, the method further comprises outputting said summary statistics comprising said one or more analytical insights. In some embodiments, the method further comprises generating and outputting a comparison of a group comprising said one or more subject data states to another group, population, or a reference material. In some embodiments, said comparison compares summary statistics for said group with said another group, said population, or said reference material. In some embodiments, said one or more groups of similar reference data states is determined to be most similar to said one or more subject data states. In some embodiments, the method further comprises outputting feature importance for one or more features used to determine said one or more groups of similar reference data states as being most similar to said one or more subject data states. In some embodiments, the method further comprises outputting a ranked list of reference data states selected from said plurality of reference data states most similar to said one or more subject data states. In some embodiments, the one or more groups are identified using a similarity algorithm configured to determine statistical similarity between said one or more subject data states and said plurality of reference data states. In some embodiments, said similarity algorithm is a non-machine learning algorithm. In some embodiments, said similarity algorithm is configured to calculate Euclidean distance. In some embodiments, said one or more subject data states comprises health data for one or more subjects retrieved from an electronic medical record. In some embodiments, said health data comprises a plurality of features extracted from said electronic medical record using a natural language processing algorithm. In some embodiments, said natural language processing algorithm comprises one or more rules for keyword identification, unit conversion, internal consistency, or any combination thereof. In some embodiments, said natural language processing algorithm comprises a natural language processing model configured to annotate said electronic medical record with gold standard labels. In some embodiments, the method further comprises providing a healthcare provider application comprising a healthcare provider interface configured to present a healthcare provider with said statistical summary comprising said one or more analytical insights. In some embodiments, said healthcare provider interface comprises a first plurality of portals, wherein at least one of said first plurality of portals comprises a patient context data grouping comprising a subset of said reference data states within said one or more groups. In some embodiments, at least one of said first plurality of portals comprises an outcomes navigator data grouping comprising outcome information for said subset of said reference data states within said one or more groups. In some embodiments, said outcome information comprises one or more of cancer survival, cancer progression, adverse event status, treatment status, or mortality. In some embodiments, said adverse event status comprises neutropenia, leucopenia, thrombocytopenia, fatigue, pain, mucositis, skin rash, nausea, vomiting, constipation, diarrhea, cognitive dysfunction, nerve damage, appetite loss, organ damage, or any combination thereof. In some embodiments, said patient context data grouping comprises a second plurality of portals comprising an interactive timeline of a disease of said one or more subjects, interactive radiology imaging of said one or more subjects, a medical history of said one or more subjects, a current status of said one or more subjects , or any combination thereof

Described herein are platforms and methods for treating a patient. In some embodiments of the platforms and methods described herein, a platform and method comprises one or more user interfaces and a data insights engine. In these embodiments, the platform and method comprises a data insights engine which comprises a machine learning software algorithm (or module) configured to ingest patient data and generate insights relating to patient care. In some embodiments of the platforms and methods described herein, the platforms and methods comprise custom user interfaces that comprise a healthcare provider interface and a patient interface. In some embodiments, the platforms and methods further comprise a third party interface.

Described herein is a platform configured to assist a healthcare provider in the provision of a treatment to a patient, the platform comprising:
(a) a healthcare provider application comprising a healthcare provider interface configured to present the healthcare provider with guidance regarding the treatment;
(b) a patient application comprising a patient interface configured to receive patient input from the patient; and
(c) a server processor configured to provide a data insights engine comprising:
   (i) an ingestion module for receiving:
      (1) health data of the patient;
      (2) outcome data comprising an outcome of a similarly situated patient, wherein said similarly situated patient is from one or more groupings identified based on said health data; and
      (3) the patient input;
   (ii) an analysis module for determining a patient related insight based on the health data, the outcome data, and the patient input; and
   (iii) a presentation module for presenting the insight within the healthcare provider interface.

In some embodiments, the healthcare provider interface comprises a first plurality of portals. In some embodiments, at least one of the first plurality of portals comprises a patient context data grouping comprising the health data. In some embodiments, the health data is retrieved from an electronic medical record of the patient. In some embodiments, the patient context grouping comprises a second plurality of portals. In some embodiments, at least one of the second plurality of portals comprises an interactive timeline of a disease of the patient. In some embodiments, at least one of the second plurality of portals comprises interactive radiology imaging of the patient. In some embodiments, at least one of the second plurality of portals comprises a medical history of the patient and a current status of the patient. In some embodiments, at least one of the first plurality of portals comprises an outcomes navigator data grouping comprising the outcome data. In some embodiments, the outcomes navigator data grouping comprises a second plurality of portals. In some embodiments, at least one of the second plurality of portals comprises data relating to the similarly situated patient. In some embodiments, the data relating to at least one similarly situated patient comprises a treatment regimen of the at least one similarly situated patient. In some embodiments, at least one of the second plurality of portals is configured to present published data relating to the patient. In some embodiments, the data insights engine comprises a publication module for selecting the published data that is most relevant to the patient and for presenting the published data within the healthcare provider interface. In some embodiments, at least one of the first plurality of portals comprises an outcomes data grouping that comprises a second plurality of portals. In some embodiments, at least one of the second plurality of portals comprises a comparison of a performance of the healthcare provider to a performance of other healthcare providers. In some embodiments, the patient interface comprises a plurality of portals. In some embodiments, at least one of the plurality of portals comprises a format for entering the patient input. In some embodiments, the data insights engine comprises a machine learning software module. In some embodiments, the similarly situated patient comprises a patient sharing one or more of the following with the patient: (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, (15) treatment goal, (16) socioeconomic factors, and (15) confounding factors such as but not limited to institution or provider.

Described herein is a platform configured to provide treatment to a patient comprising:
(a) a healthcare provider application comprising a healthcare provider interface configured to present data that assists the healthcare provider in providing the treatment to the patient;
(b) a patient application comprising a patient interface configured to receive patient input from the patient regarding the treatment;
(c) a third party interface configured to receive outcome data relating to the outcome of the treatment; and
(d) a data insights engine comprising an algorithm configured to generate a statistical summary of one or more similarly situated patients from one or more groupings or states identified based on said health data that assists the healthcare provider in providing the treatment to the patient.

In some embodiments, the healthcare provider interface comprises a first plurality of portals. In some embodiments, at least one of the first plurality of portals comprises a patient context data grouping comprising health data of the patient. In some embodiments, the health data is retrieved from an electronic medical record of the patient. In some embodiments, the patient context data grouping comprises a second plurality of portals. In some embodiments, at least one of the second plurality of portals comprises an interactive timeline of a disease of the patient. In some embodiments, at least one of the second plurality of portals comprises interactive radiology imaging of the patient. In some embodiments, at least one of the second plurality of portals comprises a medical history of the patient and a current status of the patient. In some embodiments, at least one of the first plurality of portals comprises an outcomes navigator data grouping comprising the outcome data. In some embodiments, the outcomes navigator data grouping comprises a second plurality of portals. In some embodiments, at least one of the second plurality of portals is configured to present data relating to at least one similarly situated patient identified by the data insights engine. In some embodiments, the data relating to at least one similarly situated patient comprises a treatment regimen of the at least one similarly situated patient. In some embodiments, at least one of the second plurality of portals is configured to present published data relating to the patient. In some embodiments, the data insights engine generates the data that assists the healthcare provider in providing the treatment to the patient by analyzing the patient input. In some embodiments, at least one of the first plurality of portals comprises an outcomes data grouping that comprises a second plurality of portals. In some embodiments, at least one of the second plurality of portals comprises a comparison of a performance of the healthcare provider to a performance of other healthcare providers. In some embodiments, the patient interface comprises a plurality of portals. In some embodiments, at least one of the plurality of portals comprises a means for entering the patient input. In some embodiments, the similarly situated patient comprises a patient sharing one or more of the following with the patient: (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, (15) treatment goal, (16) socioeconomic factors, and (15) confounding factors such as but not limited to institution or provider. In some embodiments, the third party comprises a payer and wherein the third party interface comprises a performance of a payer network that is determined based on the outcome data.

Described herein is a computer implemented method for treating a patient comprising:
(a) ingesting with a machine learning software module:
   (i) health data of the patient;
   (ii) outcome data comprising an outcome of a similarly situated patient, wherein said similarly situated patient is from one or more groupings identified based on said health data; and
   (iii)a patient input regarding the treatment;
(b) analyzing the health data, outcome, data, and patient input thereby generating an analysis result;
(c) generating a treatment insight based on the analysis result; and
(d) presenting the treatment insight within a healthcare provider application.

In some embodiments, the healthcare provider application comprises a healthcare provider interface comprising one or more portals. In some embodiments, the health data is retrieved from an electronic medical record of the patient. In some embodiments, the outcome data comprises an outcome of a treatment regimen. In some embodiments, the patient input regarding the treatment comprises a request to modify one or more of a treatment plan, treatment regimen, or treatment goal. In some embodiments, the similarly situated patient comprises a patient sharing one or more of the following with the patient: (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, (15) treatment goal, (16) socioeconomic factors, and (15) confounding factors such as but not limited to institution or provider.

### BRIEF DESCRIPTION OF THE DRAWINGS

The novel features of the invention are set forth with particularity in the appended claims. The file of this patent contains at least one drawing/photograph executed in color. Copies of this patent with color drawing(s)/photograph(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 shows an illustrative diagram comparing the traditional approach (left) of generating multiple custom models for generating individual predictions from a data set and the data insights approach (right) of some embodiments of the present disclosure by which a single model can be used to identify patient groupings from which insights can be determined.

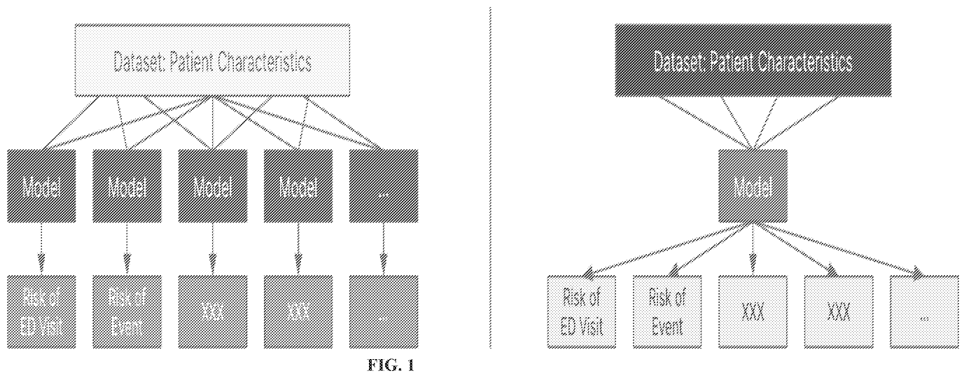

FIG. 2 shows an overview illustrating a patient state being compared to reference patient states to identify one or more groupings of similar states.

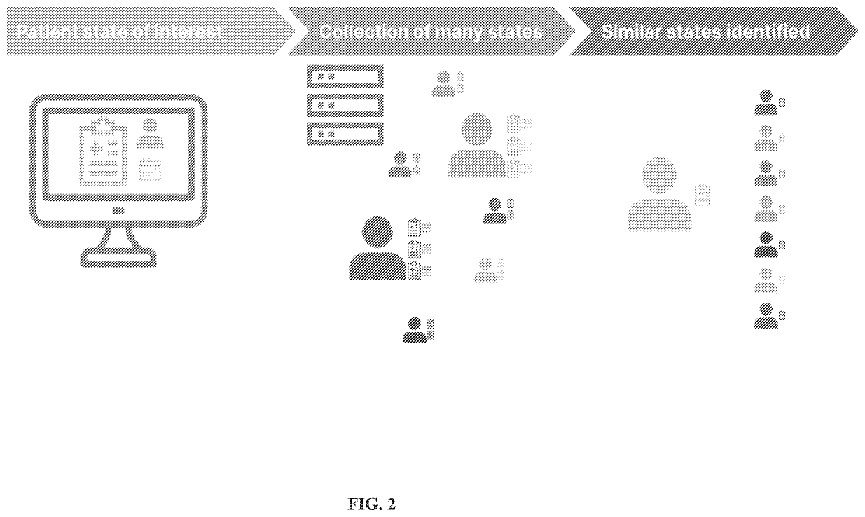

FIG. 3 shows an illustrative overview of similarity determination by minimum Euclidean distance.

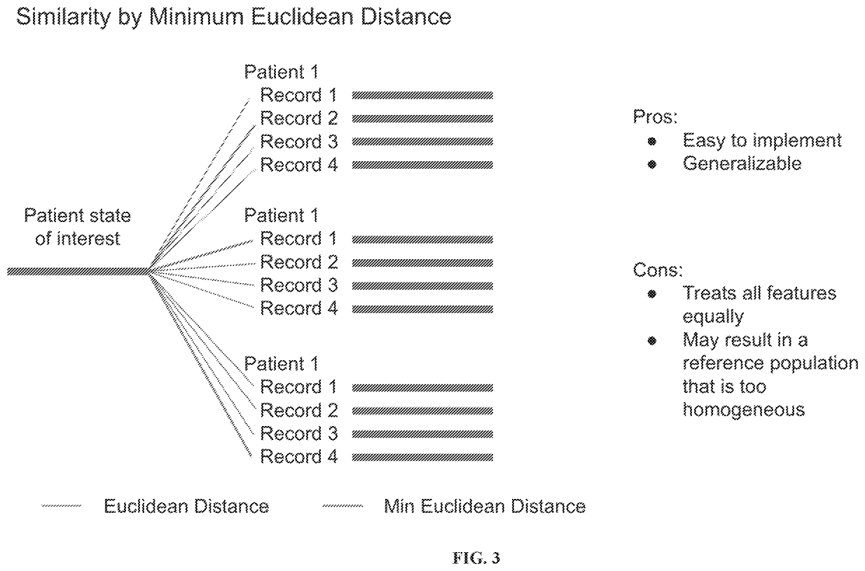

FIG. 4 shows an illustrative overview of similarity determination by treatment course block.

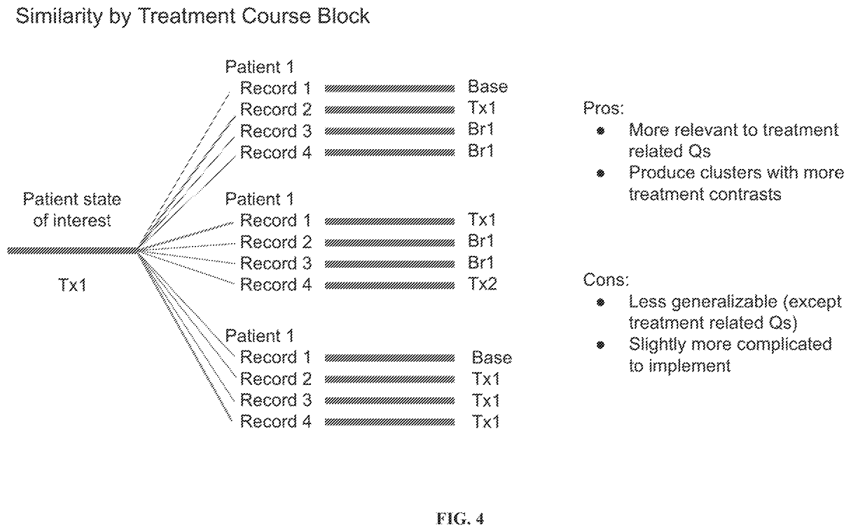

FIG. 5 shows an overview of the comparative insights generation process in accordance with some embodiments.

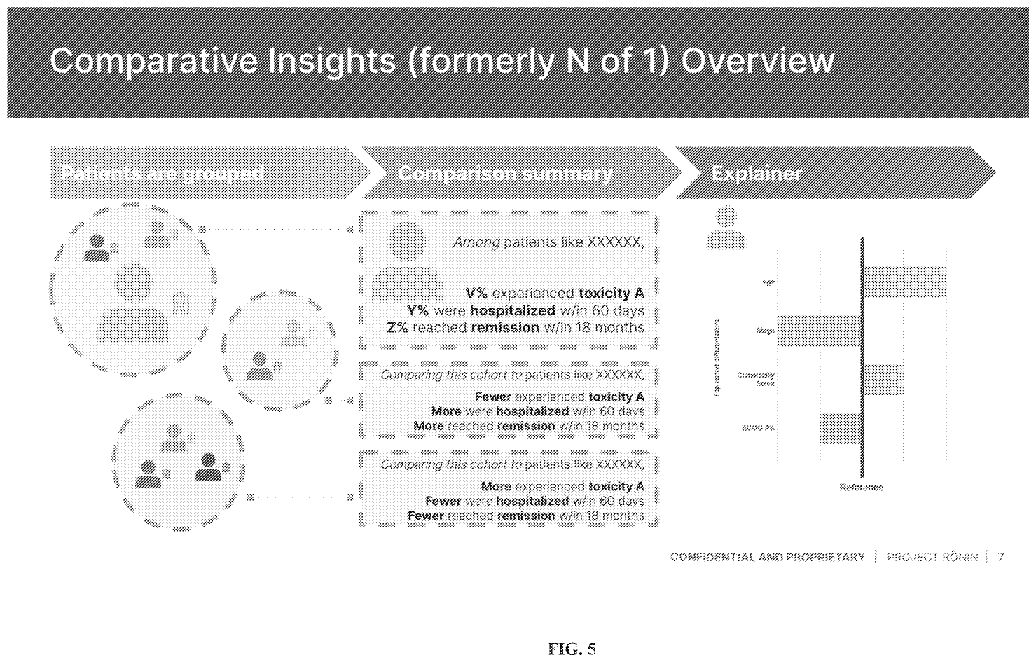

FIG. 6A shows a diagram illustrating the use of an encoder and decoder for dimensionality reduction.

FIG. 6B shows a diagram illustrating the use of a neural network encoder and a neural network decoder in accordance with some embodiments.

FIG. 7 shows an example of a digital processing device; in this case, a device with one or more CPUs, a memory, a communication interface, and a display.

FIG. 8 shows an example of a web/mobile application provision system; in this case, a system providing browser-based and/or native mobile user interfaces.

FIG. 9 shows an example of a cloud-based web/mobile application provision system; in this case, a system comprising an elastically load balanced, auto-scaling web server and application server resources as well synchronously replicated databases.

FIG. 10 shows an example of a data extraction process that incorporates natural language processing to standardize data inputs for data processing and analysis using predictive models.

FIG. 11 shows a performance comparison of the data insights engine utilizing a variational autoencoder for generating comparative insights with alternative approaches.

FIG. 12 shows an illustration of a hypothetical patient's journey through a latent feature space over time.

FIG. 13 shows an illustrative example of a user interface dashboard.

FIG. 14 shows a diagram comparing the relative performance and transparency of machine learning predictions.

FIG. 15 shows an illustrative timeline with classification or grouping into low, medium, and high percentile groups.

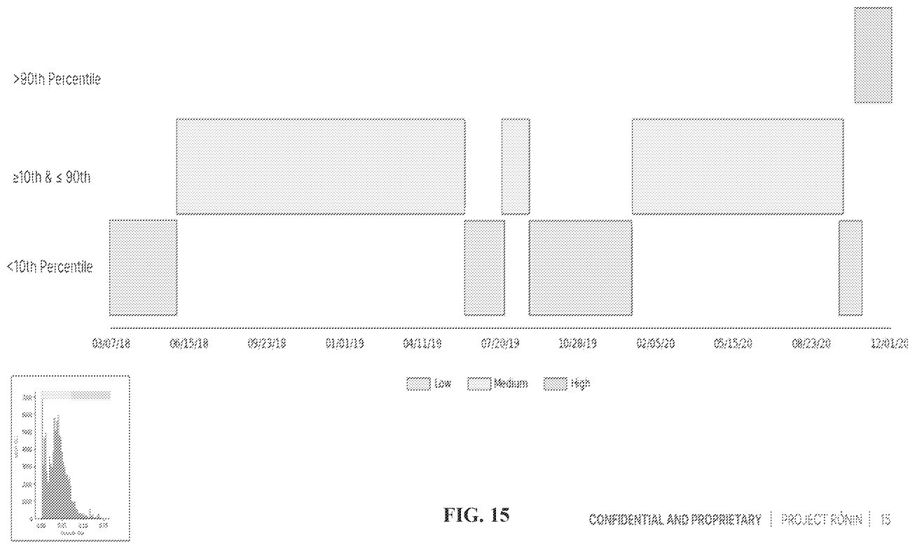

FIG. 16 shows an illustrative example of a user interface dashboard. A timeline of an acuity index ranging between high, medium, and low is shown. Events (e.g., hospitalization), treatments, patient reported symptoms, laboratory test results, and vitals are also shown.

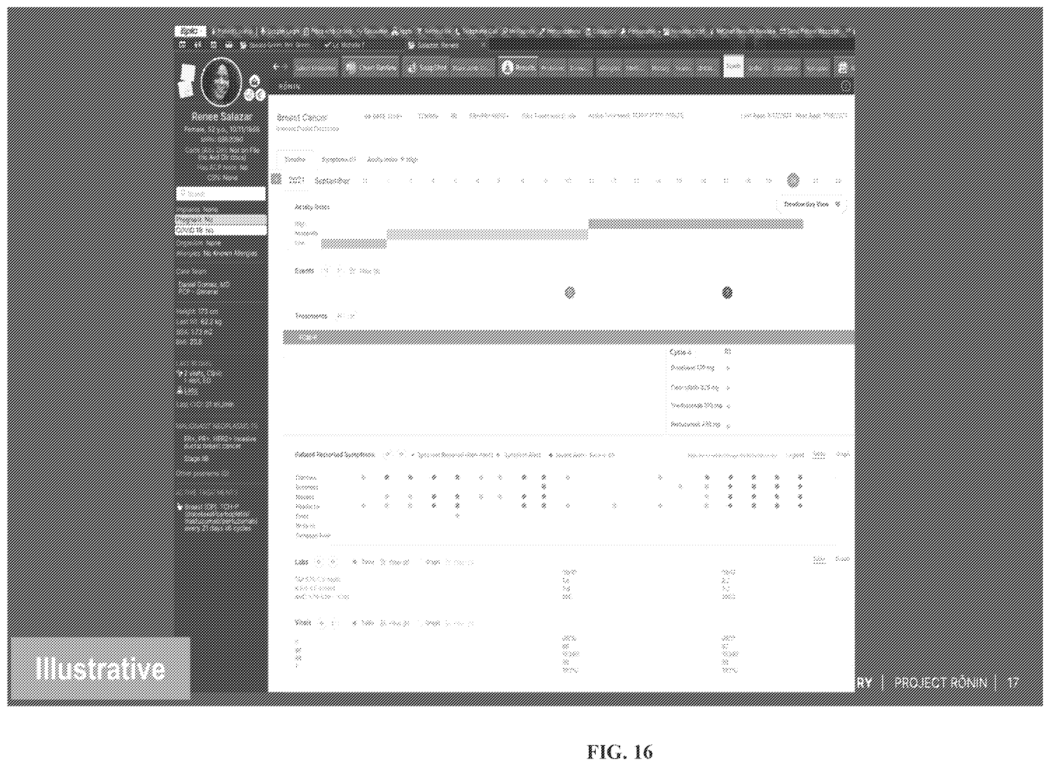

FIG. 17 shows an illustrative example of a user interface dashboard.

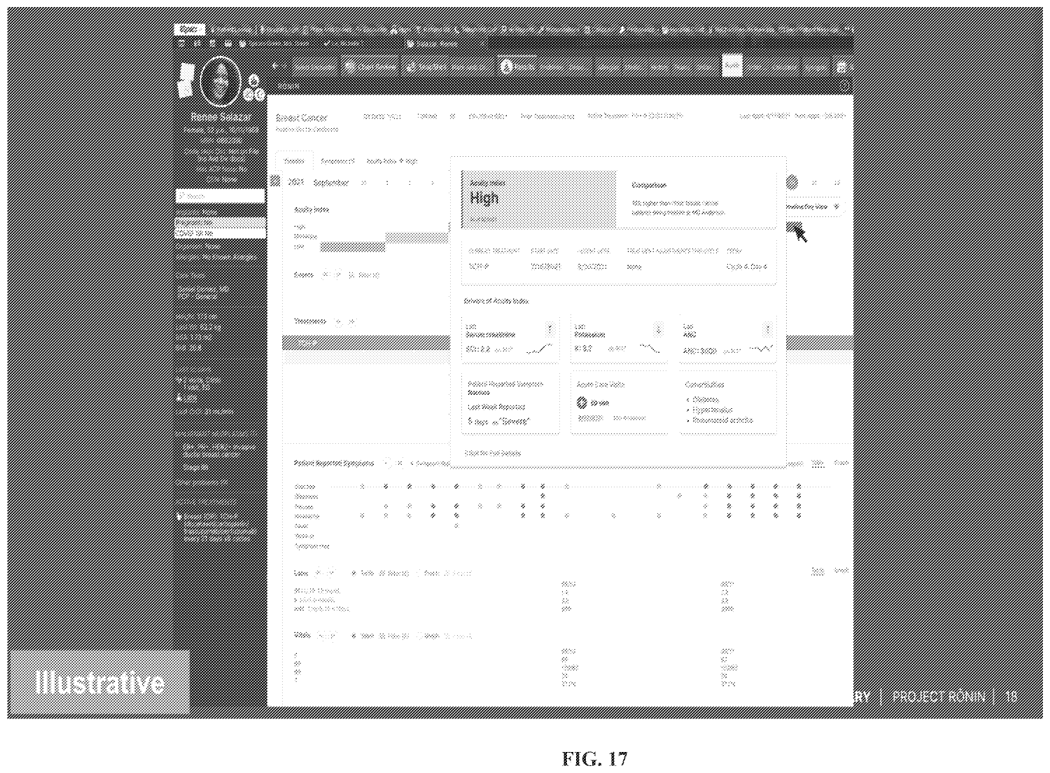

FIG. 18 shows an illustrative example of a user interface dashboard. The acuity index feature shown on the dashboard is selected. The details for the acuity or risk index is shown indicating the number of patients analyzed at the specific cancer department in the same facility and the subset of patients having similar characteristics that were used as a reference for this patient. A listing of attributes driving the risk or acuity index is shown, including those relating to socioeconomic/demographic (age, marriage status), treatment (immunotherapy, supportive medications), cancer (TNM classification), comorbidities (diabetes, hypertension, rheumatoid arthritis), and lab results (serum creatinine, potassium, ANC). A patient comparison breakdown based on the 200 similar patients is shown indicating the subset within the similar patients that were matched based on particular attributes (e.g., treatment, genomic results, performance status, age, stage and TNM, prior or concurrent treatments, histology, comorbidity, active medication).

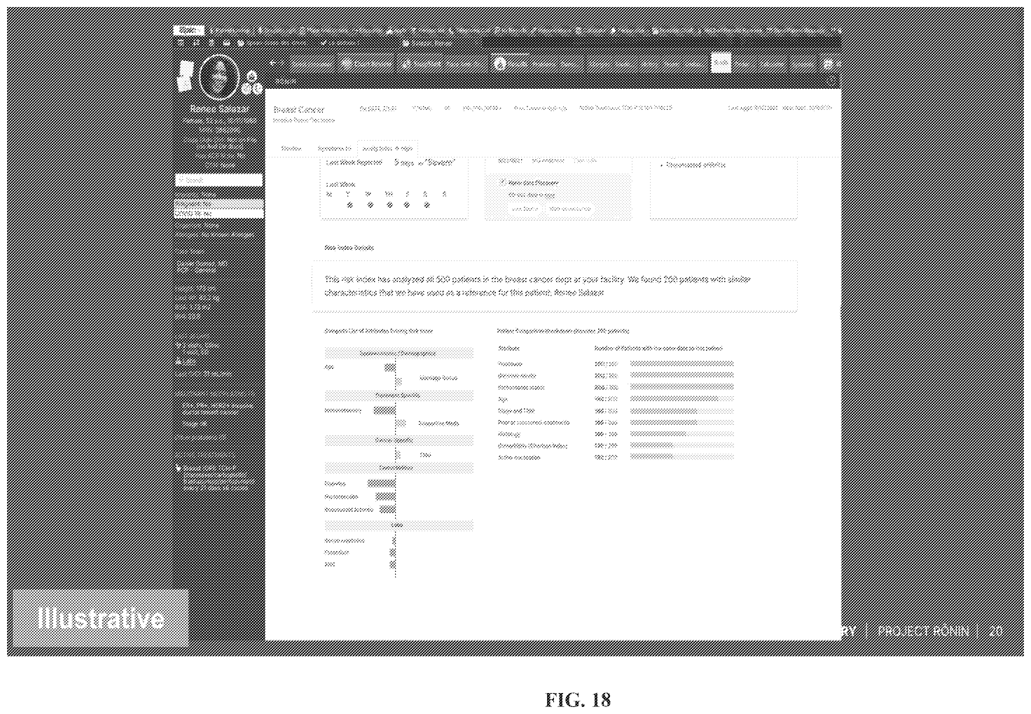

FIG. 19 shows an illustrative example of a user interface dashboard with a population level view. This view shows a breakdown of the number of patients and the subsets that fit within different risk categories (low, moderate, high risk). Related insights are also shown with interactive buttons/features that can be selected to explore those insights in more detail. The illustrative insights shown in FIG. 19 include the patients who are most likely to experience severe adverse events, the patients most likely to be hospitalized within 60 days, and the patients most likely to visit the emergency department.

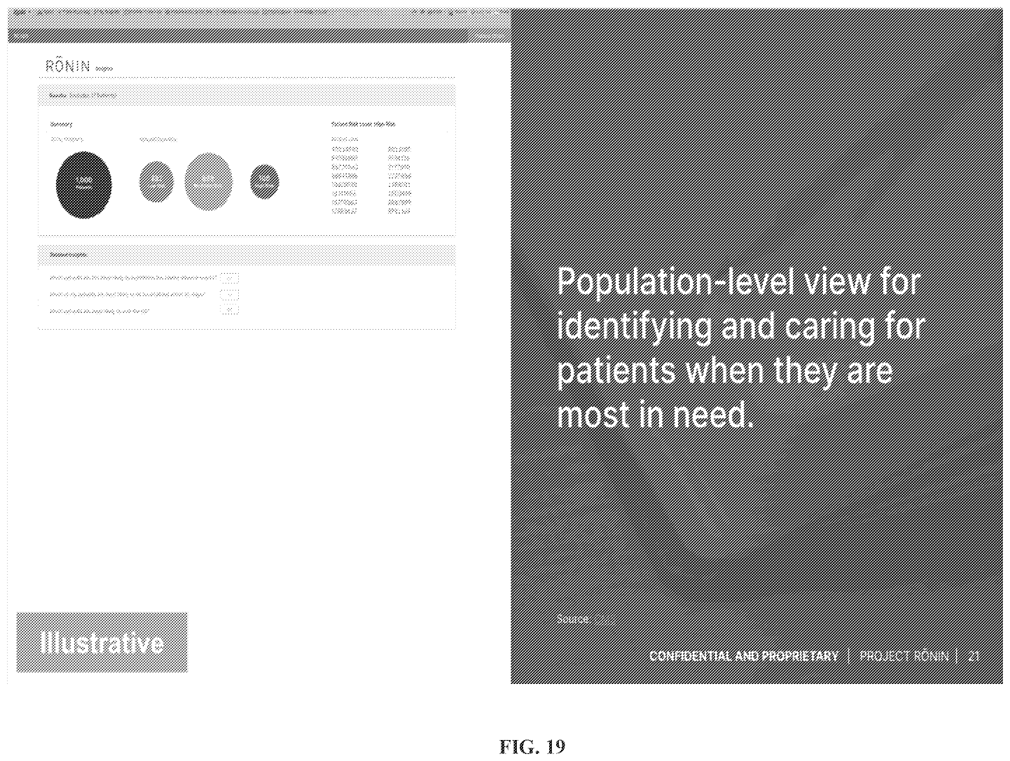

FIG. 20 shows an illustrative example of a user interface dashboard. In this example, a query was entered asking what adverse events were experienced by patients with similar characteristics. The dashboard shows the results identifying 122 patients, and a percentage listing of the top adverse events (neutropenic fever, acute kidney injury, rash, diarrhea, nausea, and shortness of breath) and the top severe adverse events. Similar patient characteristics from the similar patients are also shown. Suggestions of related questions are shown including likelihood the patient will visit the emergency department within 6 months, and what common next line treatment options are available for similar patients.

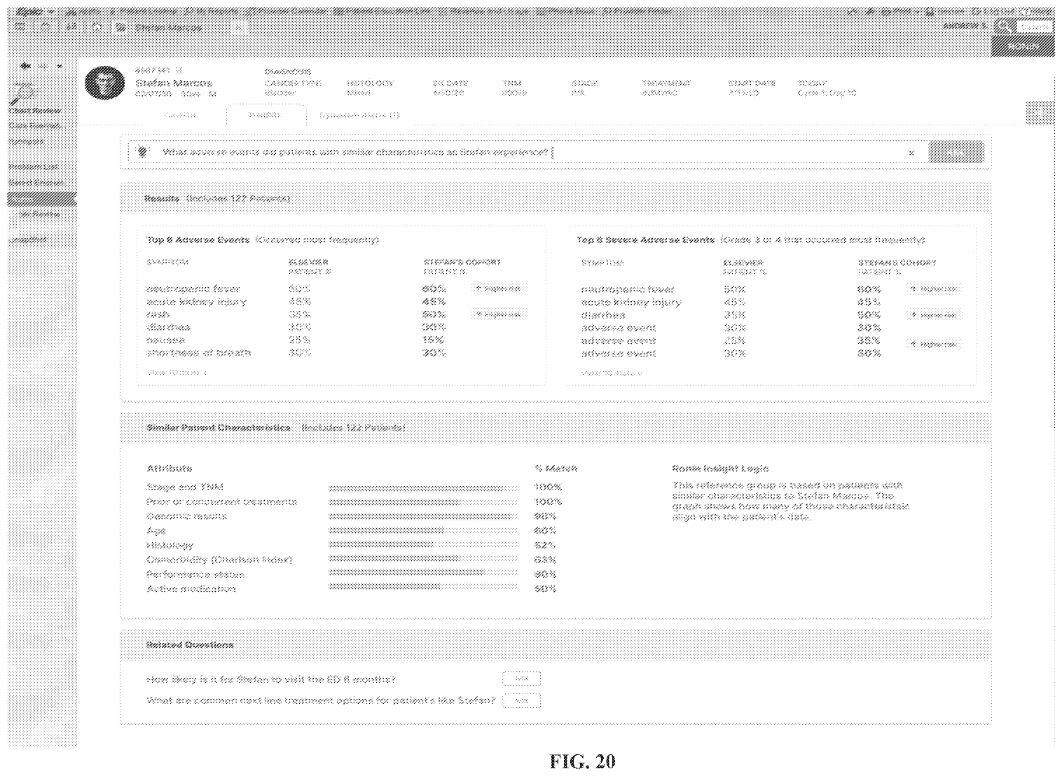

FIG. 21 shows an illustrative example of data analysis and guidance of healthcare decision making according to embodiments of the present disclosure. A course correction showing potential options for changing treatment is shown, which are based on different goals such as changing to a different therapy based on effectiveness and tolerability, a dosage reduction based on safety focus and commonality, or no change to the treatment combined with supportive care based on efficacy focus.

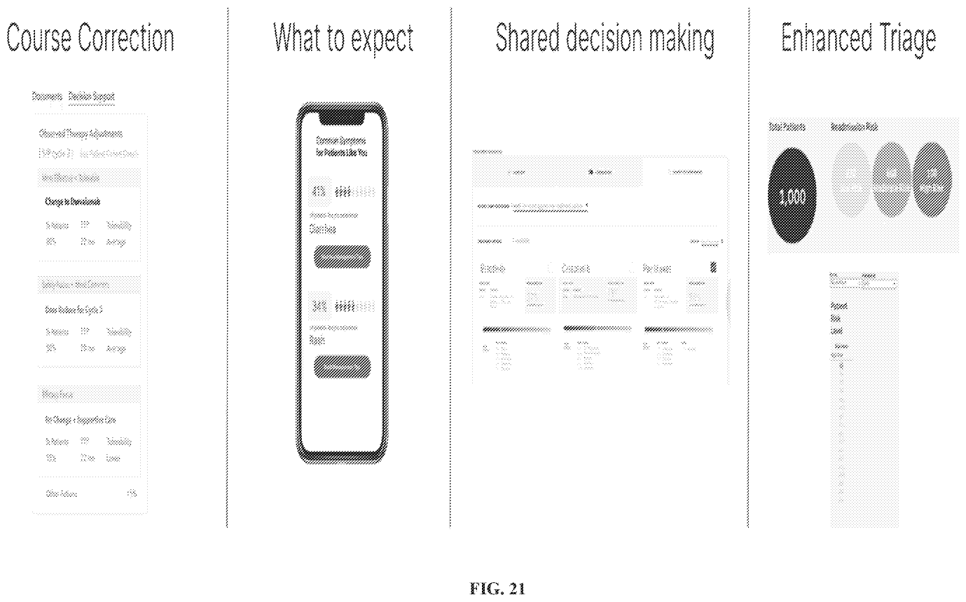

FIG. 22 shows a flow chart diagram illustrating an example of the process for generating summary statistics as described herein.

### DETAILED DESCRIPTION OF THE INVENTION

Described herein are platforms and methods configured to provide treatment to a patient. While a platform as described here in embodiments is configured for treatment of oncology patients (i.e., patients with a cancer diagnosis), the embodiments of the platform described herein is useful in providing treatment to non-oncology patients as well.

While some exemplary embodiments described herein are described with reference to oncology patients, these exemplary embodiments are not meant to be limited to the treatment of oncology patients only.

### Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise.

As used herein, a “treatment regimen” includes any medicament or medical procedure that could be used to treat a patient. Non-limiting examples of medicament types include chemotherapies, pain medications, anti-nausea medications, and antibiotics. Non-limiting examples of medical procedures include radiation therapy, surgery, physical therapy, and psychological therapy. A treatment regimen further includes within its scope any dosing of medicaments or scheduling of medical procedures to be provided to an individual. For example, a treatment regimen may include providing a chemotherapeutic agent twice a week to a patient at a certain dose per body weight of the patient. For example, a treatment regimen may include scheduling a surgery to excise a tumor followed by one month of once weekly chemotherapy. That is, a treatment regimen typically includes the details of the provision of a medicament and/or procedure in terms of, for example, quantities, frequency, order, and duration.

As used herein, a “treatment plan” includes a plan and organization of treatment with respect to a goal that is typically selected by both the healthcare provider and patient. For example, a treatment plan may be configured to provide the goal of curative treatment irrespective of side effects or treatment risks. Such a plan may be, for example, selected for younger patients with cancer types that are either curable or typically have longer periods of remission. For example, a treatment plan may be configured to provide the goal palliative care. Such a treatment plan may be, for example, selected for an older patient with a difficult to treat and/or incurable cancer type and may further take into account potential side effects and patient lifestyle preferences. That is, some patients may elect to forgo a more aggressive treatment plan in order to avoid resulting side effects that could limit their chosen life quality level.

As used herein a “patient” and an “individual” are often used interchangeably. Typically, these terms refer to a human or animal in need of treatment. Typically, the reason that the human or animal is in need of treatment is to address a cancer.

As used herein a “portal” is a component of a software application configured to provide a user with a computer based format for interacting with data, wherein an interaction with data may comprise, for example, any interaction related to sight, touch, and/or sound. A portal, in some embodiments, comprises a graphic user interface. An example of a graphic user interface format for interacting with data comprises a webpage or webpage application that is, for example, html based. A portal, in some embodiments, includes a voice-recognition application for voice interaction by the user with the data associated with the portal. In some embodiments, a portal includes a software application for providing data audibly. In some of these embodiments, the portal is entirely configured for audible interaction with, for example, the data transmitted audibly to a user and a user interacting with the data through a voice recognition software application.

As used herein an “interface” is a component of a software application configured to provide a specific user with a customized computer based format for interacting with data, wherein an interaction with data may comprise, for example, any interaction related to sight, touch, and/or sound, and as used herein includes at least one portal. In addition to at least one portal, an interface, in some embodiments, includes additional customized portals as well as other content that is not provided via a portal. An interface, in some embodiments, comprises a graphic user interface. An example of a graphic user interface format for interacting with data comprises a webpage or webpage application that is, for example, html based. An interface, in some embodiments, includes a voice-recognition application for voice interaction by the user with the data associated with the portal. In some embodiments, an interface includes a software application for providing data audibly. In some of these embodiments, the portal is entirely configured for audible interaction with, for example, the data transmitted audibly to a user and a user interacting with the data through a voice recognition software application.

As used herein a “similarly situated patient” is a patient who shares some degree of commonality with another patient (such as, for example, a patient being treated by a healthcare provider who is using a platform as described herein). Similarity can be computed based on comparison of patient states as described herein. Non-limiting examples of features that a similarly situated patient may share with a patient being treated include (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, (15) treatment goal, (16) socioeconomic factors, and (15) confounding factors such as but not limited to institution or provider (e.g., healthcare provider such as a hospital). It should be understood that with respect to some features, a similarly situated patient may not have the exact identical feature as another patient but may instead be similar to that feature. For example, within the scope of a similarly situated patient determined to be similarly situated based on age is, for example, a similarly situated patient who is 2 years younger than a patient being treated.

### Need for an Oncology Patient Specific Platform

Described herein is a platform configured to improve the existing way in which healthcare is provided to patients. While the platforms described herein are configured to improve the existing way in which healthcare is provided to all patients, there is a particular need for such a platform with respect to oncology patients.

Namely, the existing manner in which healthcare is provided to oncology patients is among other things:
(1) overly complex in terms of decision making,
(2) does not assist healthcare providers in decision making or treatment provision,
(3) is not sufficiently data-driven,
(4) does not sufficiently solicit or take into account patient feedback, and
(5) does not effectively provide useful data to healthcare payers and pharmaceutical developers.

### Existing Treatment Decision-Making is Overly Complex

Currently, the existing treatment decision-making pathway for healthcare professions is overly complex. That is, the process of choosing the proper (1) treatment regimen (which includes, for example, any medicament or medical procedure that could be used to treat a patient as well as dosing of medicaments or scheduling of medical procedures to be provided to an individual) and (2) treatment plan (which includes, for example, a plan and organization of treatment with respect to a goal that is typically selected by both the healthcare provider and patient) for a patient is extremely complex, and as a result healthcare providers are struggling to provide proper care to their patients.

The complexity around selecting one or more of a treatment regimen and treatment plan in today's existing healthcare paradigm is at least in part due to the (1) relatively large amount of information that a healthcare provider must process in order to reach the optimal decision and (2) the unique nature of every cancer and every patient.

With respect to selecting treatment regiments, in the field of oncology in particular, there are a large amount of new therapeutics and modalities being developed and made publicly available on a regular basis. New drugs are constantly being developed and studied and old drugs are being deemed less efficacious. Surgery in some cases is recommended and in other cases, new studies may reveal that non-surgical treatment is superior or that surgery with adjuvant therapy is best. And, sometimes study findings seem to contradict one another. That is all to say, that with respect to selecting an optimal treatment regimen for a patient, healthcare providers must themselves keep up with constantly newly developed treatment modalities being released and then sort through a barrage of constantly newly generated study data in order to determine which modality serves his or her patients best. In the field of oncology in particular, this is an extremely difficult task. Add to that, the fact that it is generally believed that “no two cancers are alike” in the sense that, for example, even the same cancer type may have very differently behaving subtypes and each patient may behave differently with the same cancer due to, for example, their age or genetic makeup. Additionally, there is constantly changing data with respect to dosing and the provision of adjuvant therapy along with surgery. Not uncommonly, there is no single regimen that suits all patients, but rather it is often the case that each patient needs an individualized treatment regimen. For example, some patients, due to suffering strongly from side effects, do not tolerate a recommended treatment modality and will need to have a custom treatment regimen developed.

With respect to selecting treatment plans, there is often imperfect data available as to how to customize a particular treatment to achieve a specific healthcare provider and/or patient goal. Sometimes, there is imperfect communication between the healthcare provider and the patient as to what the treatment goal is.

The platform described herein, in contrast, is configured to at least address the above issues in order to simplify and optimize the existing overly complex treatment decision-making process for healthcare providers treating their patients. As will be described, the instant platform is configured, in some embodiments, to at least (1) help healthcare providers navigate the overwhelming amount of data available to them with respect to healthcare related decisions and (2) address the unique nature of each cancer and each patient.

Existing Treatment Software Applications Do Not Provide Sufficient Assistance to Healthcare Providers in terms of Decision Making

Existing treatment software applications do not provide sufficient assistance to healthcare providers in terms of decision making. As described herein, an “existing treatment software application” includes any computer based application, software, platform, and/or system configured to provide a user interface for healthcare providers that is configured to assist the healthcare providers in terms of patient decision making. That is, existing treatment software applications do not (1) aggregate data and/or (2) digest and present data in a manner that is useful to healthcare providers.

For example, existing treatment software applications do not have the ability to aggregate large amounts of data, where data includes patient data from Electronic Medical Records (EMR) as well as data generated by clinical studies. The typical existing treatment software application platform is either lacking in patient EMR data or lacking in clinically derived data or both. As such, healthcare providers often must find data that they need to assist with their decision making on their own, and at least in the case of patient EMR data, are often limited in terms of what they can access.

For example, even when existing treatment software applications have access to certain types of data, they are typically unable to process the data and present it in a manner that actually assists healthcare providers in terms of decision making. That is, often the data aggregated by the existing treatment software applications is presented in a form that doesn't assist the healthcare provider in terms of his or her decision making because, for example, the existing treatment software applications do not make clear how the presented data relates to a particular individual being treated by the healthcare provider.

The platform described herein, in contrast, is configured, in some embodiments, to aggregate large amounts of data and, in some embodiments, to analyze aggregated data using advanced analytics and machine learning in order to provide healthcare providers with information that will directly assist the healthcare provider in terms of decision making.

### Existing Healthcare Decision Making is Not Sufficiently Data Driven

Existing decision making processes are not sufficiently data driven. That is, oncologists typically do not frequently consider data when making treatment decisions. In part, this is due to the issues already described in terms of the complexity of the current paradigm as well as healthcare providers not having access to data because of an absence of data and inadequate data presentation. Additionally, some of the lack of application of data in decision making also arises from healthcare providers not trusting data or believing that data should not necessarily be consulted when making healthcare related decisions.

In some embodiments, the instant platform creates an interface for healthcare providers that integrates data (and in particular aggregated and analyzed data) into the interface in a manner that healthcare providers trust and are willing to incorporate into their decision making. In some embodiments, a platform as described herein provides a healthcare provider with patient information relating to similarly situated patients to the patient the healthcare provider is treating. In these embodiments, the platform provides specific outcome data for the therapy pathway selected for the similarly situated patient.

### Existing Treatment Does not Sufficiently Consider Patient Feedback in Decision Making

Existing healthcare provided decision making does not sufficiently consider patient feedback. For example, with oncology patients in particular, treatment may take place over a relatively long period of time, as opposed to a discrete treatment, and patient needs may change. Often it is the case that the treating healthcare provider initiates a treatment regimen for the patient and does not waiver from the treatment regimen, while the patient may in fact have a need for the healthcare provider to do so. For example, a patient may not have realized the severity of the side effects associated with a particular treatment regimen and desires that it be modified. For example, the patient may have an event during their treatment regimen that they wish to attend and that would require them to pause their regimen. Another example of when a patient's input may not be sufficiently considered is with respect to the initial choice of the treatment regimen wherein a patient may wish to achieve a certain goal with the treatment and the healthcare provider fails to match the treatment regimen for the patient with the goal that the patient wishes to achieve.

The instant platform, in contrast, in some embodiments, provides a patient interface in the form of, for example, a portal in which the patient is asked to provide feedback that is used by the platform to guide the healthcare provider. In this manner, the platform, in these embodiments, is integrating patient input into how the platform interacts with the healthcare provider in order to incorporate patient feedback and needs into the healthcare decision making process.

Existing Treatment does not Effectively Provide useful data to Healthcare Payers and Pharmaceutical Developers

There is a need for improved communication of information from the clinics to third parties such as payers and pharmaceutical and medical device developers. Often times, third parties must wait to obtain data from studies, which creates a lag time between the outcomes to patients and the arrival of the data to the third party.

In contrast, the instant platform, in some embodiments, includes third parties within the platform so that they are able to access data directly as well as input data of their own into the platform.

### Platform Overview

In general, a platform as described herein is configured to assist a healthcare provider in providing treatment to a patient. More specifically, a platform is configured to assist a healthcare provider in selecting a treatment for a patient. In some embodiments, a platform is also configured to assist a healthcare provider in executing a selected treatment for a patient. In some embodiments, a platform is further configured to assist a healthcare provider in diagnosing a patient.

In general, a platform as described herein comprises a computer implemented or software based system that comprises one or more user applications. For example, in some embodiments, a user application comprises a healthcare provider application which is configured to be used by a healthcare provider.

A platform, in some embodiments, includes a patient application.

In some embodiments, a platform includes a third party application, where a third party is a party involved in providing healthcare to patients. Non-limiting examples of third parties that may have a third party application integrated with embodiments of the platforms described herein include healthcare payers, pharmaceutical developers, medical device developers, research institutions, and hospitals.

In general, a platform as described herein comprises a processing component configured to ingest data, process data, and output an insight regarding said data or an insight related to a patient.

A processing component, in some embodiments, comprises software having one or more modules configured to carry out one or more of the tasks of (1) ingesting data, (2) processing data, and (3) outputting an insight regarding said data, an insight related to a patient, or an insight relating to both. In some embodiments, software suitable for use with a platform as described herein comprises a machine learning algorithm.

### User Inquiry and Intent Extraction

In some embodiments, a platform, system, device, media, or method disclosed herein comprises a user input module configured to receive a user input such as an inquiry or question. The input can be fed into an algorithm configured to extract the intent of the user input for determining how to respond and/or what summary statistics or analytical insights are to be generated. This platform allows a user to type a free-form inquiry or question from which one or more intents are extracted. The intent can include or define at least one target variable, exclusion criteria, a type of comparison, or any combination thereof The type of comparison can be an individualized comparison or a group comparison, for example, comparing a patient to another patient, comparing a patient to a group of patients, or comparing a group of patients to another group of patients. In some cases, the type of comparison is a ranking, for example, a subset of patient states from a cohort that the query patient state has been grouped that is ranked according to degree of similarity to the query patient state. As an example, for a patient included within a particular group, the outcomes of that group can be compared to epidemiologic data, patients with the same cancer or treated in the same department at the same hospital, drug inserts, or to published results in medical literature. The insight in this example is that the relative risk of the patient (e.g., this patient group is twice as likely to have severe nausea than the average breast cancer patient), which is easier for humans to interpret than absolute risk (55% of patients like the patient had severe nausea).

These intent(s) form the basis for the type of analysis to be performed by the insights engine. For example, a user may enter an inquiry stating, “what adverse event could I experience if I undergo chemotherapy?”. The algorithm can utilize natural language processing and an ontology module to determine the intents within this inquiry to include “risk of adverse event” and “chemotherapy treatment”. In some embodiments, intents are classified using rules. For example, for Adverse Events, the algorithm may look for ae, adverse event, or side effect. Alternatively, intent classification can be performed using some supervised model.

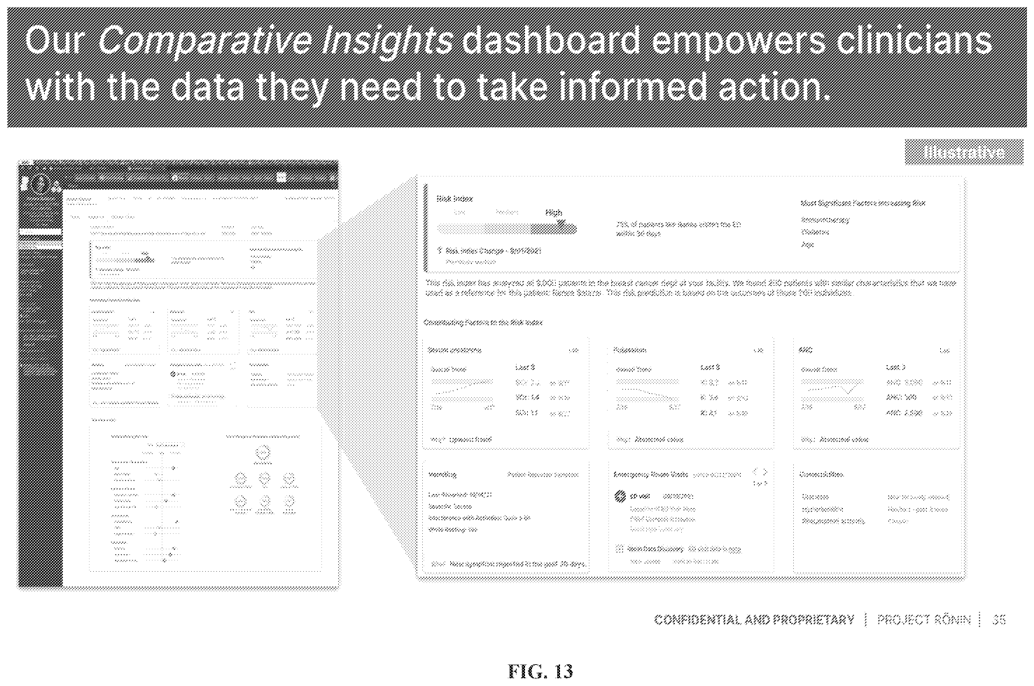

A comparative insights platform can provide a user interface accessible at a remote computing device through which a user enters inquiries such as free text questions which are translated into intents using NLP/NLU. In some embodiments, the user interface comprises a drop down menu or bar that the user could interact with to select the outcome(s) to be predicted. In some cases, the outcome to be predicted is predefined by the user interface. FIG. 13 shows an illustrative embodiment of a user interface comprising a comparative insights dashboard for showing summary statistics and insights for the patient based on a group of similar reference patient states. As shown in the illustrative user interface or dashboard of FIG. 13, a risk index is shown (e.g., low, medium, high risk) and an explanation stating that a percentage of individuals like this patient have visited the emergency department within 30 days (e.g., based on a state comparison according to the patient's current state). Risk factors that contribute or increase this risk may be shown, for example, immunotherapy, diabetes, and age. An explanation for the summary statistics/insights may be provided. As an illustrative, example, the explanation can indicate how many patient were analyzed to arrive at the risk index, where the analyzed patients were located, the number of patients with similar characteristics that were used as a reference for this particular patient, and what the risk prediction was based on (e.g., the patients used as a reference). The contributing factors to the risk index can include laboratory test results (e.g., abnormal serum creatinine, potassium, or ANC levels), patient reported symptoms (e.g., vomiting, ER visits), and other relevant factors (e.g., comorbidities such as diabetes, hypertension, and rheumatoid arthritis).

In some embodiments, the intent corresponds to a template. A template is a user experience (UX) element and pertains to the way the data is displayed. For example, there can be a template UX for questions that are at a single patient level with one outcome (e.g., “what symptoms should I expect when I start treatment with Paclitaxel?” or “am I likely to visit the ER?”). Alternatively, if the inquiry/question is “what symptoms should I expect?”, there can be a corresponding template for single patient level with many outcomes. In some cases, a question such as “which of my patients are the most similar to patients that were hospitalized within 60 days?” could yield template for a population view and/or ranked patient view.

### Data Ingestion

In some embodiments, a platform comprises a data ingestion module configured to ingest data into the platform's processing component. In some embodiments, a platform's processing component comprises a data insights engine and an ingestion module is a component of a data insights engine.

In some embodiments, data is manually provided by an individual to the data ingestion module which is configured to receive said manually inputted data. For example, in some embodiments, a user such as a healthcare provider, patient, or third party provides data to the ingestion module by entering data into an application or portal via typing on a keyboard, keypad, and/or touch screen.

For example, in some embodiments, a user such as a healthcare provider, patient, or third party provides data to the ingestion module by transmitting the data via email, text, and/or by voice (i.e., by speaking). The ingested data can be processed and incorporated into one or more data states (e.g., subject data states or reference data states). A subject data state refers to a data state for a specific subject for whom a user input or query is provided to generate insights and/or a statistical summary. A reference data state refers to a data state for other individuals on the database that are analyzed to identify one or more cohorts of data states that are statistically similar to the subject state(s).

In some embodiments, a data ingestion module is configured to either retrieve or receive data from one or more data sources, wherein retrieving data comprises a data extraction process and receiving data comprises receiving transmitted data from an electronic source of data.

For example, some embodiments of the platforms described herein are configured to retrieve or receive data from many different data sources such as wearable devices, EMR providers, and DNA providers. In some embodiments, the platform is configured to ingest data from a plurality of wearable devices and applications, and many health providers that are hosted on EMR vendors, non-limiting examples of which include Epic, Cerner, AllScripts, AthenaHealth, and VA (HealtheVet), as well as the raw genome-wide genotyping data provided by direct-to-consumer DNA labs including, by way of examples, 23andMe, Ancestry.com, MyHeritage, and FamilyTreeDNA. In some embodiments, coding system data such as, for example, ICD-9/10 data is extracted by the ingestion module.

In some embodiments, data that is ingested by the platform is sorted based on, for example, data type. In some embodiments, the data insights engine determines how to sort the data based on various factors such as, for example, the healthcare provider and/or the patient of the healthcare provider.

In some embodiments, data that is ingested by the platform is cleaned. In these embodiments, data is cleaned in the sense that corrupt data is either corrected or deleted. Examples of corrupt data include, for example, incorrectly entered or misfiled data. For example, corrupt data to be cleaned includes typographical errors and inaccurate data.

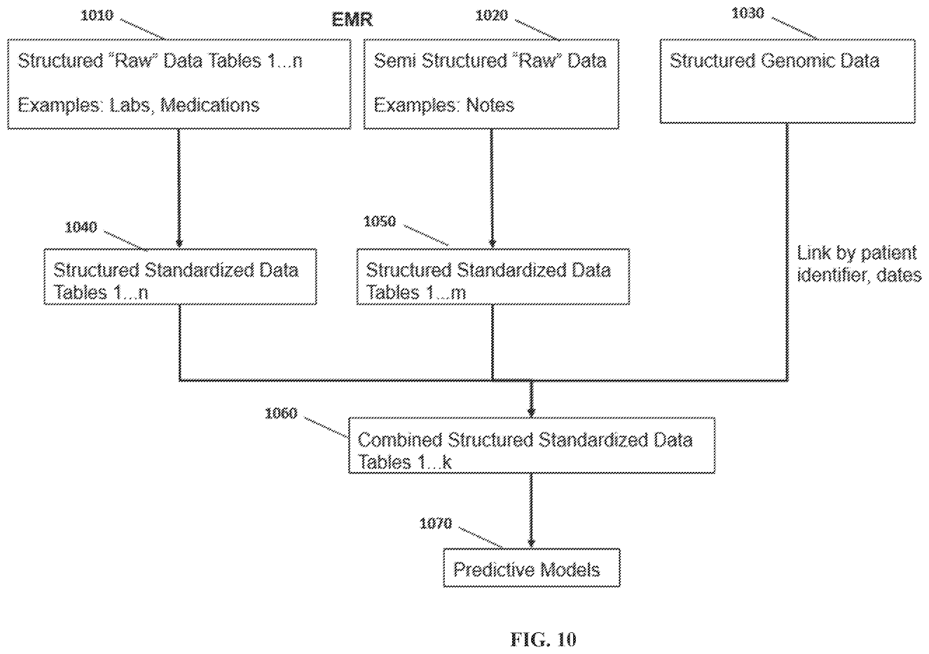

In some embodiments, data is extracted from one or more data sources. An example of a data extraction process is illustrated in FIG. 10. The data source(s) include structured raw data 1010, semi-structured raw data 1020, structured genomic or genetic data 1030, or any combination thereof. Examples of structured raw data include laboratory tests/results and medications or prescriptions, which can include information such as lab names, medication names, and other associated information. Semi-structured raw data can include less structured information such as doctor's notes from a patient consultation or treatment checkup. Structured genomic data can include results of targeted biomarker testing or large-scale tests such as whole genome sequencing. For example, the organized results of a mutation panel of the top 100 cancer genes is an example of structured genome data (e.g., a table listing the genes with corresponding mutation status).

In some embodiments, data is extracted from one or more data sources using natural language processing (NLP). NLP allows for relevant data to be extracted from free text, for example, physician notes from an electronic health record or electronic medical record. One NLP approach is rule-based NLP which applies various automated rules to standardize and/or format the sourced data into a common standard that is compatible with downstream data processing and/or analysis. For example, the structured laboratory results may be evaluated to identify keywords associated with each standardized lab name or medication name. The rules can also apply unit conversion logic or check for internal consistency, for example, cross-reference the data to determine if a medical checkup or treatment administration date is consistent across parts of the same medical record or different medical records. A detected inconsistency may be resolved using one or more rules or alternatively flagged for manual resolution. These rules may be curated by a user based on the particular domain to which the data belongs (e.g., lab test or prescription). In some cases, the application of automated rules to the data enables detection of inconsistent values that are flagged. For example, in one scenario, a treatment table shows medication X given on date A for Cycle 2 and date B for Cycle 3, but date B is before date A. In this case, the flagged error may be auto-corrected by referencing the Visit table and determining when visits were scheduled for the cycles of treatment. Accordingly, the information extracted from the structured raw data 1010 and/or semi-structured data 1020 can be converted into structured standardized data 1040, 1050.

Another NLP approach utilizes supervised machine learning to annotate one or more portions of the raw text with gold standard labels. For example, a name in the raw data may correspond to a standardized name. A training data set including labeled text is used to train a NLP ML model. As an example, NLP models can be used to extract adverse events, treatment response, and other relevant medical information from an electronic medical record (EMR) into one or more controlled or standardized vocabularies (e.g., MedDRA, RECIST). Internal consistency checks can also be performed as described herein. Accordingly, the information extracted from the structured raw data 1010 and/or semi-structured data 1020 can be converted into structured standardized data 1040, 1050.

Another NLP approach does not use gold standard labeling, but instead combines the previous two NLP approaches by generating rules based on the raw data, and then using the rules to train a model to apply to the raw data during the standardization/formatting process. Instead of training the NLP model on human annotated gold standard labels, the NLP model is trained based on agreement between the rules. An advantage of this approach is it enables models to be constructed without requiring a manual annotation process which can be time-consuming. This approach is applicable to the conversion of structured raw data 1010 and/or semi-structured data 1020 into structured standardized data 1040, 1050. Any combination of the NLP approaches disclosed herein can be used to standardize the input data received from various data sources. Various tools can be used to implement the NLP approaches described herein, including open source NLP packages such as, for example, Snorkel. As an example, clinical data expertise is used to create a large number of rules based on the observed data structure. An advantage of this approach is that the accuracy of the individual rules does not need to be high. The rules are then used to score records by applying each rule to each record, creating a matrix of rule outputs. The model is then trained iteratively via an algorithm that upweights rules that agree in their output for a plurality of records, and downweights rules that tend to disagree with other rules. The output of the model is a weighted set of rules where essentially each rule contributes a weighted vote on the best data standard for a record. Together, the weighted combination of rules form a data standardizing function that has been learned from multiple eligible component parts. Accordingly, the NLP model can be configured to effectively standardize and/or process input data even though the individual rules used to score records lack a high accuracy.

In some embodiments, data elements in free text are standardized. One approach for standardizing data elements in free text is to use Word Embeddings. Word Embeddings are low dimensional numerical representations of free text words. Words Embeddings allow performing similarity calculations between words. For example, the similarity score (or cosine similarity) between mets and metastasis could be high, but mets and diagnosis could be low when trained on a corpus of medical notes. With Word Embeddings, data elements which are not standardized, for example mets could be substituted for metastasis and can be used with other rule-based approaches as disclosed herein.

### Data Processing

In some embodiments, a processing component of a platform comprises a data insights engine. A data insights engine is generally an algorithm (or software module) that analyzes ingested data and provides an insight related to the ingested data or a patient being treated based on the analysis. In some embodiments, the data insights engine generates a statistical summary of one or more cohorts of reference data states that are most similar to a subject data state.

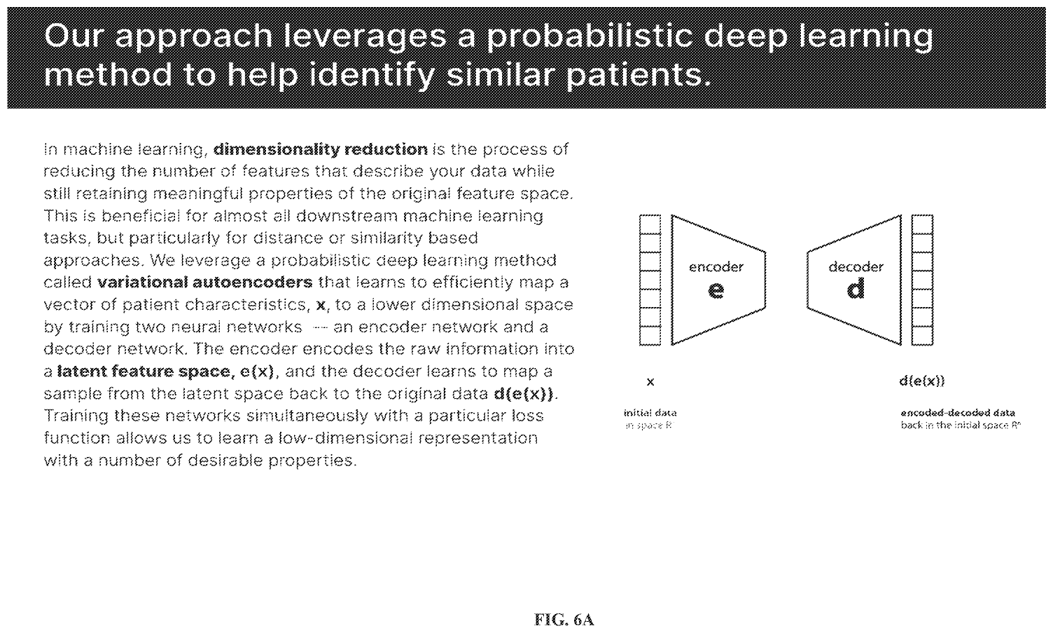

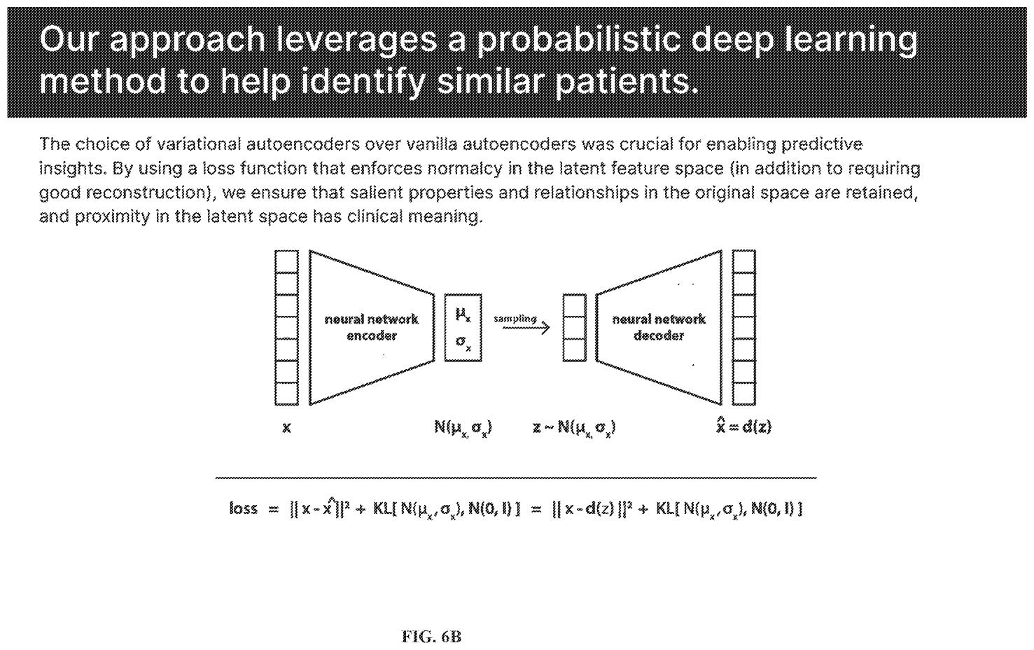

In some embodiments, information for a plurality of individuals is parsed and processed to generate a plurality of reference data states, for example, corresponding to a plurality of time points. In some embodiments, a machine learning technique is used to perform dimensionality reduction to reduce the number in features in a given data set while still retaining the meaningful properties of the original feature space. This technique is useful for various downstream machine learning tasks. In particular, dimensionality reduction is helpful for distance or similarity based downstream analyses of the data (e.g., generation of patient insights and comparisons). In some embodiments, a probabilistic deep learning method is used to perform dimensionality reduction. In some embodiments, the probabilistic deep learning method comprises the use of an autoencoder such as, for example, a variational autoencoder. FIG. 6A shows an illustrative diagram representation of a variational autoencoder. The variational autoencoder allows for the efficient mapping of a vector of patient characteristics, x, to a lower dimensional space by training two neural networks—an encoder network and a decoder network. The encoder encodes the raw information from the patient data set into a latent feature space, e(x), and the decoder learns to map a sample from the latent space back to the original data d(e(x)). Training these networks simultaneously with a particular loss function allows the generation of a low-dimensional representation with a number of desirable properties.

FIG. 6B provides another illustrative diagram representation of a variational autoencoder. The use of variational autoencoder over vanilla autoencoders can be crucial for enabling predictive insights. Using a loss function that enforces normalcy in the latent feature space (in addition to requiring good reconstruction) ensures that salient properties and relationships in the original space are retained and that proximity in the latent space has clinical meaning. Accordingly, similarity or distance between a subject patient state and one or more reference patient states can be computed based on a comprehensive view of the patient's current state. In some embodiments, a given patient state (subject and/or reference) can include social determinants of health and demographic risk factors. Examples include gender, race, ethnicity, marital status, body mass index (BMI), age, economic stability, education access, and healthcare access. A patient state can include prior healthcare utilization usage, for example, time since last visit (e.g., days since last doctor's visit), previous windowed visits, previous windowed ed visits, days since last ed, admission status, and other indicators of prior healthcare utilization. A patient state can include comorbidities such as Exlishauser indices, Charlson comorbidity index, problems within the past 30 days, symptoms, and other related indicators. A patient state can include cancer-specific risk factors such as PSA (prostate-specific antigen), IDH mutation, beta-HCG, AFP, TNM, cancer type, drugs, additional lab results, and treatment_rxnorm. Some of these data points are NLP-derived, for example, previous windowed ed visits, days since last ed, admission status, Elixhauser indices, and symptoms. Some of these data points are obtained via geo-mapping, for example, economic stability, education access, and healthcare access. Some of these data points are graph-based, for example, cancer type, drugs, and treatment_rxnorm.

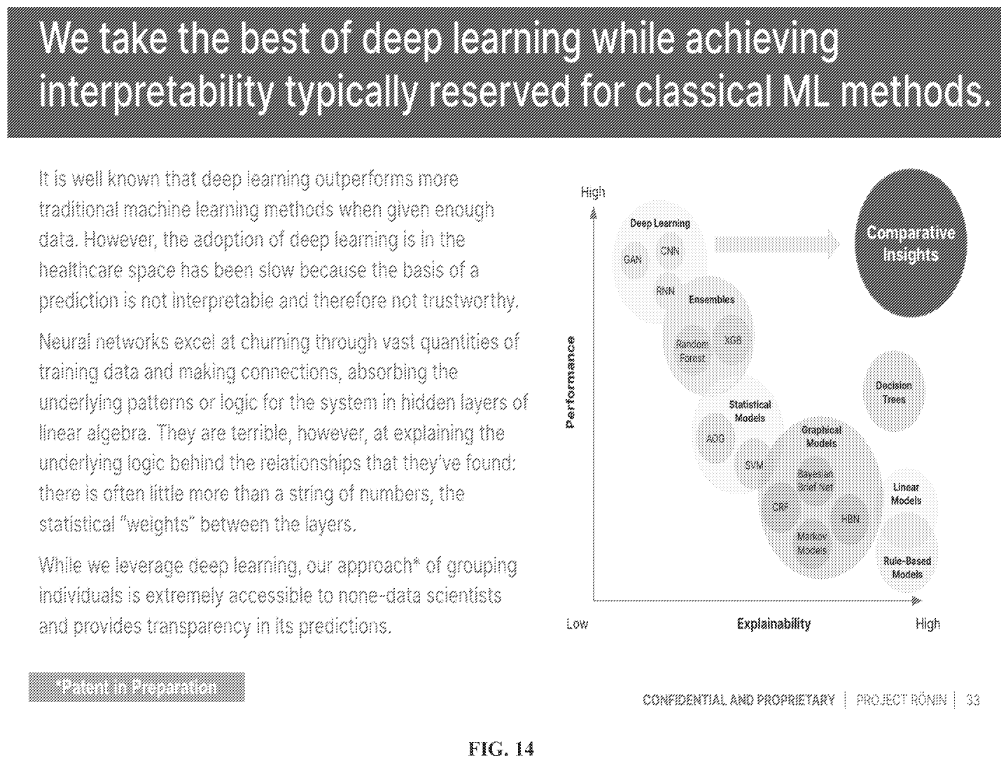

The approach of using deep learning for purposes of grouping individuals provides a technical solution to the problem of generating accurate insights that also provide transparency in the prediction. For example, as illustrated in FIG. 14, while deep learning is known to outperform more traditional machine learning methods when given enough data, the adoption of deep learning in healthcare has been slow because the basis of a prediction is not interpretable (i.e., “black box”) and therefore not considered trustworthy. Neural networks excel at processing vast quantities of training data and making connections, absorbing the underlying patterns or logic for the system in hidden layers of linear algebra. However, neural networks fail to explain the underlying logic behind the relationships that they have uncovered, and there is often little more than a string of numbers, the statistical weights between the layers that convey these relationships. By contrast, the present disclosure leverages deep learning for grouping individuals that allow for predictive or comparative insights to be generated with transparency. For example, features that significantly contributed to a patient being included in a particular grouping can be identified and displayed via the user interface (e.g., comparative insights dashboard). FIG. 14 provides an illustration of the relative performance and explainability or transparency of the comparative insights generated according to one embodiment of the data insights engine adopting a variational autoencoder compared to other approaches.

The plurality of reference data states can be structured into an index table based on the intents extracted from a user query. In some embodiments, a search such as elastic search (or other suitable search algorithm or engine) is used to generate the index table. In some embodiments, the intent(s) extracted from an inquiry are fed into an elastic search algorithm to generate an index table of patient snapshots that allows retrieval of hierarchical data.

In some embodiments, patients are grouped into non-mutually exclusive groups by a one or two-step algorithm. 1) All patient records are temporally aligned with the records of the patient being interrogated based on their medical history. A single record is selected for each patient that corresponds to the current record for the patient being interrogated based on similarity. 2) Patients are then grouped based on the similarity of a set of relevant patient characteristics or features. Dimensionality reduction is a beneficial intermediate prior to determining peer by proximity. This may include methods that enforce normality in the reduced dimensional space. This similarity is measured using metrics such as, but not limited to, Euclidean distance.

In some embodiments, additional questions can be identified as related to the current question. These additional questions can be provided as recommended questions and subsequently answered in conjunction with the current question. For example, additional questions can be determined to be frequently asked in conjunction with the current question using item-user and/or item-item collaborative filtering of similar methods.

In some embodiments, the data insights engine generates an output comprising summary statistics and/or one or more insights. In some embodiments, the data insight(s) are provided via summary statistics generated from one or more groups of similar reference states. The output can include summary statistics on a target variable within the one or more groups of similar reference states for an individual based on patient characteristics. In some embodiments, the output comprises a comparison of the individual's group summary statistics to another group, population, or other reference materials. Examples of such reference materials include clinical trial results (e.g., Elsevier), epidemiological data (e.g., global burden of disease, SEER), or normative datasets.

In some embodiments, the output comprises information regarding feature attribution or importance that contribute to inclusion/exclusion from the group and/or with respect to the target variable. For example, the relative importance or degree of a particular feature used to group the patient within a particular group of reference states may be important to building physician trust of the output. In some embodiments, the output comprises an overall percent match and specific patient characteristics of interest (e.g., features contributing to the group inclusion/match).

In some embodiments, the summary statistics and feature importance are generated to compare two individuals or groups of individuals, which may result in a ranked list of individuals with regard to the target variable. In some embodiments, the output comprises a counterfactual explanation. For example, a counterfactual explanation may indicate or describe an alternative output (e.g. a different grouping) if the characteristic(s) of the individual of interest had been different.

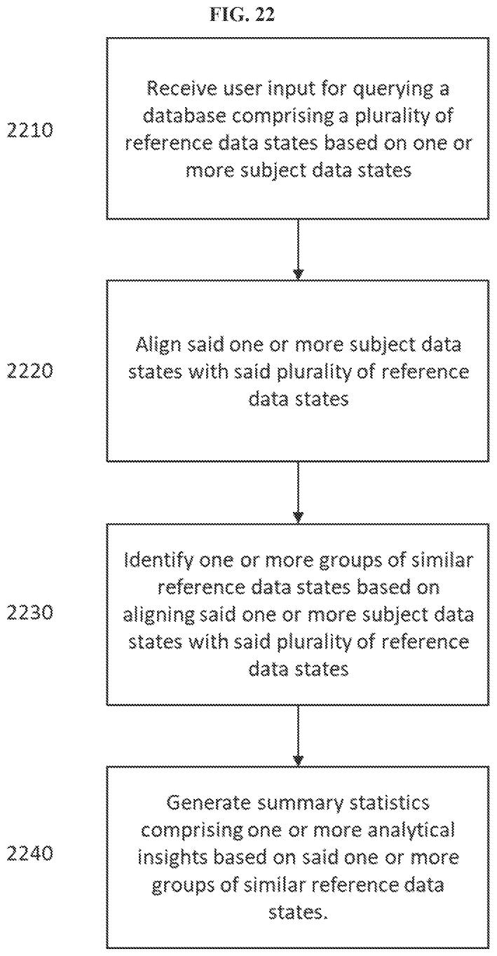

FIG. 22 provides an illustrative example of a process for generating summary statistics. The flow chart shows steps comprising receiving user input 2210, aligning the subject state with reference states 2220, identifying similar reference states based on the alignment 2230, and generating summary statistics based on the identified similar reference states 2240. This diagram is for illustrative purposes only and is not intended to limit the systems and methods disclosed herein, and additional steps not shown in the diagram may be performed.

In some embodiments, a data insights engine comprises artificial intelligence software or a machine learning algorithm (or software module).

In some embodiments, the data insights engine comprises one or more machine learning algorithms or models configured to generate insights. Insights can include outcomes such as patient survival (e.g., 5-year survival), progression (e.g., degree of cancer progression), adverse event (e.g., neutropenia), or other relevant outcome metrics. The outcome can relate to a cancer such as, for example, colorectal cancer, breast cancer, lung cancer, prostate cancer, hepatocellular cancer, gastric cancer, pancreatic cancer, cervical cancer, ovarian cancer, liver cancer, bladder cancer, urinary tract cancer, thyroid cancer, renal cancer, carcinoma, melanoma, or brain cancer. The outcome may be related to one or more therapeutic treatments. The treatment can include one or more of chemotherapy, hormone therapy, targeted therapy, radiation therapy, stem cell transplant, surgery, or immunotherapy.

In some embodiments, the adverse event comprises hospitalization caused by one or more side effects of a particular treatment or therapy. In some embodiments, the adverse event comprises discontinuation of the therapy caused by one or more side effects. In further embodiments, the one or more side effects are selected from the group consisting of: neutropenia, leucopenia, thrombocytopenia, fatigue, pain, mucositis, skin rash, nausea, vomiting, constipation, diarrhea, cognitive dysfunction, nerve damage, appetite loss, organ damage, and any combination thereof. In some cases, the adverse event comprises a serious adverse event such as death, a life-threatening side effect, hospitalization, disability, or permanent impairment or damage.

The one or more machine learning algorithms or models configured to predict individual outcomes can be implemented using a variety of model types. Model types suitable for this analysis include random forest, gradient boosted trees, penalized linear regression, penalized logistic regression, cox regression, and recurrent neural network. A machine learning model can be trained using a training data set comprising patient information labeled with outcomes. The trained model can then generate one or more predictions for the individual outcome(s). Each outcome may be ranked according to predictive confidence or accuracy. The data insights engine can identify the highest ranked outcome. In some cases, the engine outputs an insight comprising the highest ranked outcome.

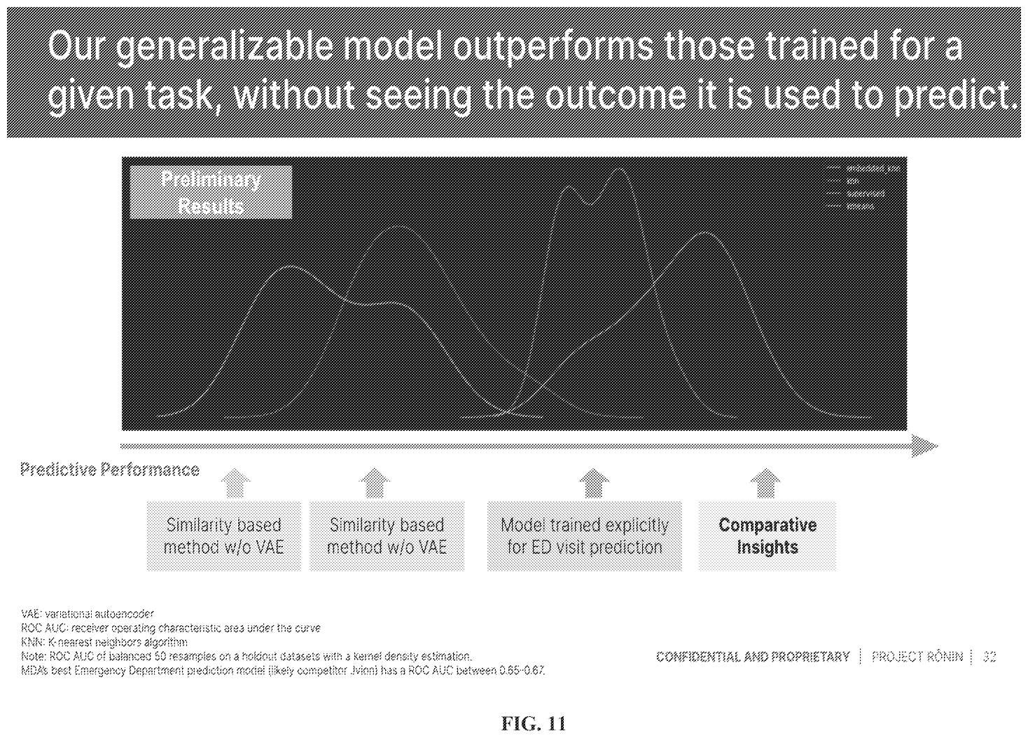

In some embodiments, the data insights engine comprises one or more machine learning algorithms or models configured to generate insights or predictions based on one or more groups of reference data states. In some embodiments, a predictive model is configured to identify insights such as predicted outcomes. FIG. 11 shows a comparative chart illustrating the predictive performance of one embodiment of a data insights engine that uses a variational autoencoder to generate comparative insights alongside alternative methods. As shown on FIG. 11, similarity methods that calculated similarity without a variational autoencoder (VAE) (kmeans and KNN/K-nearest neighbors algorithm) and a model trained explicitly for past ED visit (supervised) were outperformed by the variational autoencoder. In this example, the ROC AUC of balanced 50 resamples on a holdout datasets with a kernel density estimation. By comparison, MDA's best Emergency Department prediction model has a ROC AUC between 0.65-0.67.

In some embodiments, insights and predictions are derived based on the clinical outcome(s) of a patient's peers. These peers are individuals who are located near the patient of interest in the latent feature space. This allows for a single model to be trained on the data sets for those peers (e.g., similar reference patient states or features derived therefrom) to help answer downstream clinical questions. For example, a user may enter an input inquiry asking what is the outcome of standard chemotherapy for treating their cancer. A group of most similar reference states is identified for that user based on their cancer type and other relevant features. This group is then used to train a single model that predicts for outcome of chemotherapy.

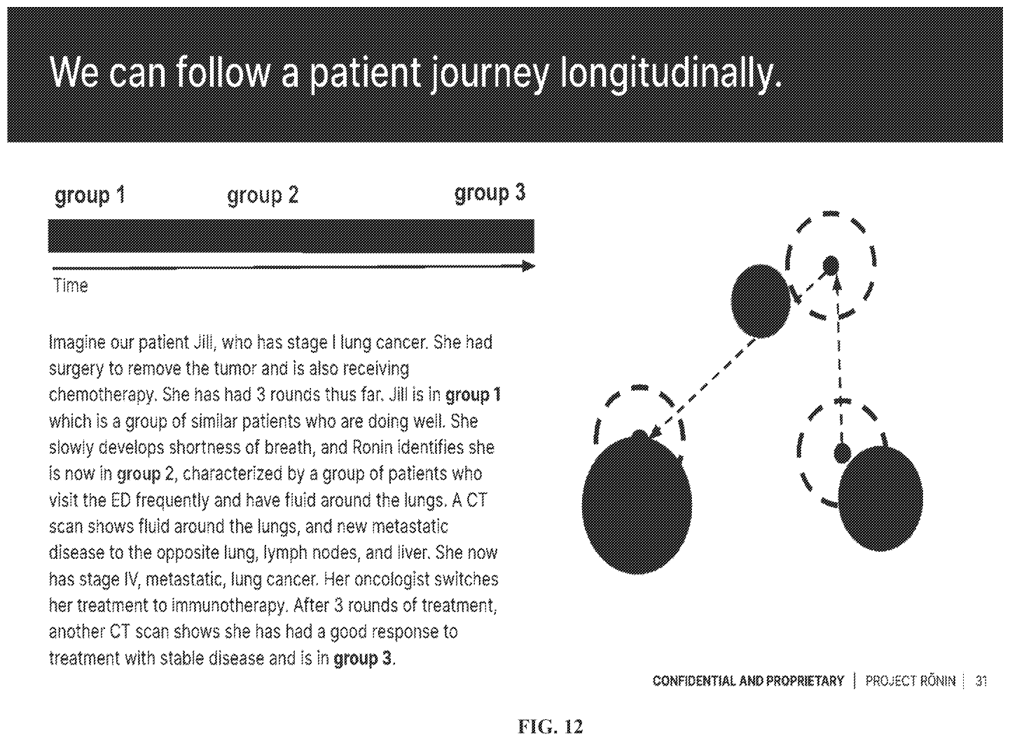

FIG. 12 provides a diagram illustrating the latent feature space and a hypothetical patient's journey through this latent feature space. In this hypothetical example, a patient has stage I lunch cancer and has had surgery to remove the tumor and is also receiving chemotherapy. This patient has had 3 rounds of chemotherapy so far. Currently, the patient is in a group of similar patients who are doing well (group 1). Slowly, the patient develops shortness of breath, and the algorithm now groups the patient within group 2, which is characterized by a group of patients who visit the emergency department (ED) frequently and have fluid around the lungs. A CT scan shows fluid around the lungs, and new metastatic disease has spread to the opposite lung, lymph nodes, and liver. The patient now has stage IV, metastatic lung cancer. The patient's oncologist switches treatment to immunotherapy. After 3 rounds of treatment, another CT scan shows good response to treatment with stable disease, and the patient is now placed in group 3 corresponding to their updated status.

In some embodiments, a machine learning algorithm (or software module) of a platform as described herein utilizes one or more neural networks. A neural network is a type of computational system that can learn the relationships between an input data set and a target data set. A neural network is a software representation of a human neural system (e.g., cognitive system), intended to capture “learning” and “generalization” abilities as used by a human. In some embodiments machine learning algorithm (or software module), the machine learning algorithm (or software module) comprises a neural network comprising a convolutional neural network. Non limiting examples of structural components of embodiments of the machine learning software described herein include: convolutional neural networks, recurrent neural networks, dilated convolutional neural networks, fully connected neural networks, deep generative models, and Boltzmann machines.

In some embodiments, a machine learning software module comprises a recurrent neural network software module. A recurrent neural network software module is configured to receive sequential data as an input, such as consecutive data inputs, and the recurrent neural network software module updates an internal state at every time step.

In some embodiments, a machine learning software module comprises a supervised or unsupervised learning method such as, for example, SVM, random forests, clustering algorithm(s) (or software module(s)), gradient boosting, logistic regression, decision trees, and/or hidden Markov models. In some embodiments, the data insights engine comprises a hidden Markov model for use in generating predictive insights.

In some embodiments, a machine learning software module comprises a neural network comprising a CNN, RNN, dilated CNN, fully connected neural networks, deep generative models and deep restricted Boltzmann machines.

In some embodiments, a neural network is comprised of a series of layers termed “neurons.” In some embodiments, a neural networks comprises an input layer, to which data is presented; one or more internal, and/or “hidden,” layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data from data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments of a machine learning software module as described herein, a machine learning software module comprises a neural network such as a deep convolutional neural network. In some embodiments in which a convolutional neural network is used, the network is constructed with any number of convolutional layers, dilated layers or fully connected layers. In some embodiments, the number of convolutional layers is between 1-10 and the dilated layers between 0-10. In some embodiments, the number of convolutional layers is between 1-10 and the fully connected layers between 0-10.

### Patient State Alignment

In some embodiments, the grouping identified for the patient is determined using an alignment of patient or subject states against reference data states. In some cases, the reference data states are structured within an index table that is searched using the subject state. While a conventional approach may be to simply find patients with similar features to include in the cohort, this approach fails to account for timing with respect to the patient's current state and also will not be able to meaningfully group patients once the number of features in consideration is large. In some embodiments, the patient information is parsed into a plurality of patient states that may be positioned along a timeline.

For example, one patient state may be the full set of clinical/medical information for the patient when they first visited a doctor and examined for a lump that turned out to be cancer. This could include static data that remains the same across various patient states (e.g., sex, ethnicity, and other non-changing information) as well as dynamic data that can change or be initially absent and later added (e.g., age, lab test results, adverse event, pathological change such as metastasis, initiation and/or termination of treatment, and other potentially relevant clinical information.). These patient states for the individual patient can then be analyzed for similarity with respect to a patient state data set for a plurality of other patients. The group of patient states that is highest in similarity and/or has a similarity satisfying a certain threshold can be combined together into a group of the most similar patient states corresponding to the patient and subsequently used to derive summary statistics and/or analytical insights.

In some cases, a patient can have hundreds of patient states in which each additional piece of information that is added to the patient/subject state database causes a new patient state to be generated that incorporates that additional piece of information. These patient states on the database can be utilized as the plurality of reference data states against which a specific patient-based query is conducted. An index table comprising the plurality of reference data states can be used to allow a search based on the intent(s) extracted from the patient-based query and the patient/subject data state for that specific patient. Thus, the similarity for each of these patient states with respect to a particular patient can be efficiently calculated using a suitable similarity algorithm, for example, a Euclidean distance algorithm. The most similar patient state for each patient in the data set can be grouped and then used to conduct cohort analysis to identify the cohort of most similar patients.

The conventional mechanism for alignment would be to align patients on a patient by patient basis using whatever information is available. By contrast, the instant disclosure provides an unconventional and superior approach that accounts for the patient's current state in time. Unlike clinical trials, a well-defined first time point is not available in real world data. Moreover, as the complexity of care increases, a clean baseline point becomes difficult particularly as the diversity of treatment paths diverges in complex and late stage disease.

### Data Output

In general, a data insights engine of a platform as described herein outputs an insight based on data analyzed using artificial intelligence software or a machine learning algorithm (or software module) as described herein. An insight outputted by the data insights engine generally relates to inputted data, one or more patients, or both.

In some embodiments, the insight outputted is part of a statistical summary of one or more cohorts of reference data states that are statistically similar to the subject/patient data state(s). As an illustrative example, a user query may ask a question “I want the most aggressive treatment option”. In this example, the subject data state comprises a positive cancer diagnosis, and the statistical summary provides statistical insights derived from a group of similar reference data states. The group is analyzed to identify the different treatment options and the outcomes of these treatment options within the group. The statistical insights in this example include the percentage of patients within the group that were given each treatment option, the percentages of the most common side effects, and the outcome of the treatment (e.g., remission, disease progression, recurrence).

An output of a data insights engine, in some embodiments, is presented to a healthcare provider via a healthcare provider application which may further comprise a healthcare provider interface.

An output of a data insights engine, in some embodiments, is presented to a patient via a patient application which may further comprise a patient interface.

An output of a data insights engine, in some embodiments, is presented to a third party via a third party application which may further comprise a third party interface.

For example, in some embodiments an insight generated by a data insights engine of a platform as described herein comprises an identification of a patient (or a group of patients) who is similarly situated to the patient of a healthcare provider who is using the platform (e.g., based on their corresponding reference patient state(s)). A patient who is similarly situated may be, for example, a patient with the same cancer diagnosis as the patient. A patient who is similarly situated may be, for example, a patient with the same demographic information as the patient. A patient who is similarly situated may be, for example, a patient who either completed or is undergoing the same treatment regimen as planned for the patient. It should be understood that a first patient may be deemed similarly situated to another patient such as a patient of the healthcare provider using the platform for numerous reasons and based on one or more factors and the examples presented here are not intended to be limiting. It should further be understood that a plurality of similarly situated patients may be identified in the same manner as identifying a single similarly situated patient and reference to a single similarly situated patient should not be understood to be limited to one single patient only but in some embodiments includes a plurality of similarly situated patients. Lastly, it should be understood that, in some embodiments, a similarly situated patient comprises a composite of a plurality of patients. In these embodiments, once identified, a similarly situated patient along with their health data are presented to a healthcare provider treating the patient.

Health data of a similarly situated patient presented to a healthcare provider by a data insights engine as described herein may, for example, include the diagnoses, treatment regimen, and outcome of the similarly situated patient. Non-limiting examples of health data presented to a healthcare provider by a data insights engine as described herein may, for example, include pathology reports, medicament dosing, types of diagnostic and therapeutic procedures performed, and the outcomes of said diagnostic and therapeutic procedures. For example, a diagnostic procedure may comprise a radiographic study of the similarly situated patient such as a CT scan, which may be presented to the healthcare provider by the data insights engine. For example, diagnostic procedures may comprise a laboratory study of the similarly situated patient such as blood work, the results of which may be presented to the healthcare provider. In this way, a healthcare provider is provided with examples of patients and their accompanying health data that the healthcare provider can use to assist him or her in determining or adjusting a treatment regimen for the patient. For example, a healthcare provider may select a treatment regimen for the patient based on a treatment regimen provided to a similarly situated patient that the healthcare provider deems to be most similar to the patient. In some of these embodiments, a data insights engine is configured to determine how relevant the health data of a similarly situated patient may be to said healthcare provider based on information relating to the healthcare provider's practice and/or relating to the patient. For example, in some embodiments, if a data insights engine determines that a healthcare provider is mostly treating a certain type of patient and has less experience treating a patient having the attributes of the patient, the data insights engine will determine that said health data of a similarly situated patient is relevant to the healthcare provider and provide said information to him or her. In some embodiments, said data insights engine will further determine a degree of relevance to the healthcare provider expressed as a percentage and present the percentage to the healthcare provider as well. It should be noted that in this and other embodiments, a data insights engine presents outputs that assist a healthcare provider in making a treatment decision without suggesting a specific decision for the healthcare provider to make. That is, in these embodiments, the data insights engine provides an output that provides assistance to the healthcare provider without necessarily making a specific suggestion. In this way, the healthcare provider's decision making process is enhanced through the advanced analytics of the data insights engine without having the data insights engine effectively replace the decision making process by outputting to the healthcare provider what he or she should do. Assisting the healthcare provider in the decision making process rather than pointing to a decision to be made will promote broad acceptance from healthcare providers who typically desire to maintain independence and autonomy in their healthcare related decision making processes.

In some embodiments, an insight generated by a data insights engine of a platform as described herein comprises one or more treatment regimens. For example, a data insights engine may present a treatment regimen to a healthcare provider that was deemed successful for a similarly situated patient. A treatment regimen may be deemed successful, in some embodiments, when a goal of the patient is achieved through the application of the treatment regimen. In some embodiments, a treatment regimen is presented to a healthcare provider based on data received by the data insights engine from a study. For example, a data insights engine, in some embodiments, ingests and analyzes study data from, for example, a study of the effect of a pharmaceutical provided to human patients and provides information regarding said pharmaceutical to a healthcare provider based on said analysis in order to assist said healthcare provider in treating a patient. For example, a data insights engine, in some embodiments, ingests and analyzes study data from, for example, a study of the effect of a surgical procedure provided to human patients and provides information regarding said surgical procedure to a healthcare provider based on said analysis in order to assist said healthcare provider in treating a patient. For example, a data insights engine, in some embodiments, ingests and analyzes study data from, for example, a study of outcome data of patients having, for example, a certain type of cancer or a cancer in a certain stage and provides information regarding said study to a healthcare provider based on said analysis in order to assist said healthcare provider in treating a patient. In some of these embodiments, a data insights engine is configured to determine how relevant said information may be to said healthcare provider based on information relating to the healthcare provider's practice and/or relating to the patient. For example, in some embodiments, if a data insights engine determines that a healthcare provider is providing a treatment regimen to a patient that is deemed less effective than a treatment regimen based on study data, in some embodiments, the data insights engine will determine that said information is relevant to the healthcare provider and provide said information to him or her. In some embodiments, said data insights engine will further determine a degree of relevance to the healthcare provider expressed as a percentage and present the percentage to the healthcare provider as well. The degree of relevance would be based on two factors: a) the data insights confidence in the result determined via cross validation, and b) the degree to which the patient is similar to the patients where the insight is drawn from (based on hierarchical clustering or other distance based method). Again, it should be noted that in this and other embodiments, a data insights engine presents outputs that assist a healthcare provider in making a treatment decision without suggesting a specific decision for the healthcare provider to make. In this way, the healthcare provider's decision making process is enhanced through the advanced analytics of the data insights engine without having the data insights engine effectively replace the decision making process by outputting to the healthcare provider what he or she should do. Assisting the healthcare provider in the decision making process rather than pointing to a decision to be made will promote broad acceptance from healthcare providers who typically desire to maintain independence and autonomy in their healthcare related decision making processes.

In some embodiments, an output of a data insights engine comprises a prediction of an outcome for an individual or a group of individuals. An outcome that is predicted may be, for example, a result of a treatment, a result of a disease, or associated with a treating physician or healthcare facility. Outcomes may, in some embodiments, be classified in terms of, for example, safety outcome or efficacy outcome. For example, a safety outcome may be assessed in terms of whether a treatment harmed an individual receiving it through, for example, a side effect. For, example, an efficacy outcome may be determined if, for example, a treatment prolongs a life of an individual receiving being treated beyond an expected life expectancy. In some embodiments, an outcome is predicted by determining a similarity between an individual (or group of individuals) whose outcome is to be predicted and an individual (or group of individuals) having a known outcome. In these embodiments, when an individual (or group of individuals) share certain key predictive characteristics with an individual (or group of individuals) with known outcomes, it is predicted that the individual (or group of individuals) will share the same outcome as the individual (or group of individuals) whose outcome is known. For example, a first patient having certain tumor marker is compared, in some embodiments, to a second patient having the same tumor marker and a known outcome and it is predicted that the first patient will have the same outcome as the second patient based on sharing the characteristic of having the same tumor marker.

In some embodiments, one or more machine learning software modules are used to analyze patient characteristics and identify which one or more characteristics are most impactful with respect to a prediction. That is, in some embodiments, one or more machine learning software modules determine factors including patient related characteristics that are associated with or likely result in a particular outcome. In these embodiments, identification of these factors or characteristics is used to make predictions with respect to outcomes in other similarly situated patients based presence, absence, and/or degree of presence or absence of a particular factor or characteristic.

In some embodiments, the identification of factors and or characteristics is performed by one or more software modules which, in some embodiments, comprise one or more machine learning modules. Such software modules are configured to cluster (or group) individuals (or groups of individuals) in order to identify factors (e.g., environmental or personal) and/or characteristics (e.g., personal) that are associated with or cause a particular result or outcome. In some embodiments, a data insights engine comprises (or is operatively linked to) one or more software modules (which in some embodiments comprises one or more machine learning software modules) that are configured to cluster (or group) a plurality of individuals based on outcomes (e.g., treatment outcomes and/or disease outcomes). Once clustered by outcomes, the individuals or populations within each cluster of outcomes are analyzed by the one or more software modules with respect to common factors and/or characteristics to determine which characteristics within the group of individuals clustered by common outcome is a characteristic that is associated with or determinative of the outcome shared by the individuals in the group/cluster. In general, in these embodiments, the approach to determining factors and characteristics useful in making predictions is achieved by first achieving the grouping of outcomes as described herein. It should be understood that there are numerous methods suitable for achieving grouping or clustering of outcomes in order to identify factors and/or characteristics associated with or that result in an outcome.

An exemplary approach to the clustering (or grouping) of individuals with similar outcomes is an insights engine that comprises or is operatively coupled with a software module that applies a hierarchical clustering technique to a data set. Hierarchical clustering in general is a process wherein distances between data points to be clustered (e.g., outcomes) are determined and wherein data points that are determined to be closest to each other are clustered.

A hierarchical clustering technique, in some embodiments, first identifies at least two data points (e.g., outcomes) that are closest to one another and clusters them together. In a next step, it determines a distance from a third data point to the clustered original two data points. If that third data point is closest to the clustered original two data points, it will cluster that third data point with the first two. If on the other hand, that third data point is closer to a fourth data point than it is to the cluster of the first two data points, the third and fourth data points are clustered and a distance is determined between the first cluster (the first two data points) and the second cluster (the third and fourth data points). Ultimately, a hierarchical clustering technique determines distances between all the data points being analyzed and establishes a hierarchical relationship among all of the data points based on distances that are respectively apart from one another. A dendrogram (or cluster tree) is one type of visual representation of hierarchical relationships between data points in a hierarchical clustering algorithm.

In hierarchical clustering, a distance metric determines the clusters. In some embodiments, a distance metric is set by a user. In some embodiments, a distance metric is set by the algorithm itself When the algorithm itself sets the distance metric for the data points, in some embodiments, the algorithm is a learning algorithm that determines the distance metric for particular data based on previous learning/training.

In some embodiments, a software module as described herein utilizes a hierarchical technique that groups individuals (or populations of individuals) based on an outcome for the individual (or population of individuals) and the outcome comprises an efficacy of a treatment. For example, in some embodiments, an outcome comprises a survival time following the initiation of a treatment. In this manner, an outcome represents an efficacy of a treatment with respect to the treatment's effect on survival time. An outcome, in some embodiments, comprises a safety metric associated with a treatment. For example, an outcome, in some embodiments, comprises a specific side effect or plurality of side effects relating to a treatment including, for example, nausea, fever, headache, and weight loss. In this manner, an outcome represents a safety of a treatment with regard to side effects related to or resulting from the treatment.

As described, in a hierarchical clustering technique, outcomes are grouped together based on a distance metric. An example of a distance metric that might be set includes a period of time. For example, where an outcome to be clustered comprises a survival rate following the initiation of a treatment, a distance metric comprises, in this exemplary embodiment, a single year. In this example, patients who survived 5 years following treatment initiation may be grouped together because they all survived within the same year (i.e., one year which is the distance metric) from one another following the initiation of treatment. In this example, a group of patients that survived 4 years following treatment initiation may be the nearest group within the hierarchy of groupings because the second group (4 year survival) is just one year (i.e., the distance metric) apart from the first group (5 year survival). A dendrogram of clusters formed using this metric would show these two groups, 5 and 4 year survivals, and their relationships to other groups clustered based on survival from treatment initiation (e.g., a 3 year survival group). It should be understood that other durations are suitable for use with the clustering technique described herein including durations lasting any number of years including, for example, 1, 2, 3, 4, 5, 6, 7, 8, 9, or 10 years, durations lasting any number of months including, for example, 1, 2, 3, 4, 5, 6, 7, 8, 9, 10, 11, or 12 months, durations lasting hours including, for example, 1, 2, 3, 4, 5, 6, 7, 8, 9, 10, 11, 12, 13, 14, 15, 16, 17, 18, 19, 20, 21, 22, 23, or 24 hours.

In another exemplary embodiment, an outcome comprises an efficacy outcome which, in this exemplary embodiment, comprises a survival time following initiation of a treatment. Non-limiting examples of a data metric suitable for use in this exemplary embodiment include pain level following initiation of treatment (e.g., as indicated on a subjective scale), weight gain following initiation of treatment, increased appetite following initiation of treatment (e.g., as indicated on a subjective scale), changes in radiographic data (e.g., a measure of change of tumor size), or changes in vital signs following initiation of treatment.

In another exemplary embodiment, an outcome comprises a safety outcome which, in this exemplary embodiment, comprises side-effects experienced following initiation of a treatment. Non-limiting examples of a data metric suitable for use in this exemplary embodiment include total number of side effects reported, degree of severity of at least one side effect (e.g., as indicated on a subjective scale), degree of weight loss, or degree of loss of productivity (e.g., as measured by number of days absent from work).

In an exemplary embodiment of an insights engine configured to output a prediction regarding an outcome of a patient as described herein, data that is clustered using a hierarchical clustering technique (or any other suitable clustering technique) is divided into individual clusters using a modified gap statistic technique. A gap statistic technique compares intra-cluster variation for different outcomes and a modified gap statistic technique uses intra-cluster variation data to divide the different clusters into smaller numbers of clusters (e.g., individual clusters). In an exemplary next step of a predictive technique that utilizes clustering of individuals or populations of individuals by outcomes as described herein, predictive models are applied to the divided outcome clusters, wherein the outcome clusters are the dependent variables and the full set of patient characteristics are the independent variables. Exemplary predictive models applied to the one or more outcome clusters include Elastic Net Multinomial Regression and Gradient Boosted Machines. Both predictive models, once trained, correctly classify patients into similar outcome clusters with >90% accuracy.

Re-clustering occurs over time, in some embodiments, when new clusters are formed or individual results are moved into new clusters in response to additional data being received. That is, an individual's data originally placed into a first cluster may be moved to a second cluster based on additional data that is received such as, for example, new test results or findings which result in a new (i.e., different) outcome prediction. Similarly, additional data received for one or more individuals may result in a totally new outcome prediction that results in a new cluster containing individuals with the totally new outcome that is predicted. Additional data received may represent changes in existing parameters or new parameters not previously available such as, for example, patient weight, patient age, patient reported pain levels, and patient reported tolerance of treatment. Additional data received may represent changes in existing diagnostic data or new diagnostic data such as, for example, radiographic data, laboratory results, and biopsy result. Additionally, new data used to make new predictions may be in the form of intermediate outcomes. For example, an intermediate outcome may comprise an initial treatment result such as an indication of a remission of a cancer. Other non-limiting examples of intermediate outcomes include onset of side effects, tumor growth or invasion, metastasis, radiation toxicity, decrease in tumor size or degree of invasiveness, and decrease in number of metastases. In general, in such embodiments, as additional parameters related to individuals become available, the additional data is used by the software described herein to run new outcome predictions that may affect how the original data is clustered (i.e., a different or new prediction may be made based on the additional data).

In some of these and other embodiments, an output of a data insights engine is presented to one or more of a healthcare provider, a patient, and/or a third party through one or more interfaces. In some embodiments, an interface as described herein comprises a custom interface.

In some embodiments, the output comprises the feature importance or attribution for one or more features relied upon to group a subject state into a cohort of reference states or to determine the cohort of references as most similar to the subject state. It may also include counter factual explanations. Accordingly, a user such as a patient or physician can evaluate the specific features most responsible for identification of the particular cohort, which allows them to determine whether the cohort identification, summary statistics derived from the cohort, or insights based on the summary statistics are reliable. This approach can enhance user trust of the summary statistics and insights in guiding their medical decision-making, build trust with risk-averse clinicians, mitigate risk, and derive new insights.

### Interfaces

A platform as described herein typically includes different interface types customized to a specific type of user and of which there may be different numbers within the platform. For example, in some embodiments, a platform comprises healthcare provider application which comprises a healthcare provider interface, a patient application which comprises a patient interface, and a third party application which comprises a third party interface. In some embodiments, each of the healthcare provider interface, patient interface, and third party interface each comprise a custom interface that is respectively customized for a healthcare provider, a patient and a third party.

A platform in some embodiments comprises a healthcare provider application which comprises a healthcare provider interface and a patient application which comprises a patient interface, and wherein each interface is customized to a specific user, i.e., the healthcare provider and patient respectively.

A single platform as described herein may comprise one or more of any type of interface described herein. That is, in some embodiments, for example, a platform comprises a single healthcare provider application, fifty patient applications (each with a different patient/user), and two third party applications (each with a different third party). It should be understood that, in some embodiments, a single application comprises a plurality of interfaces. For example, in the previous example wherein a platform comprises a single healthcare provider application, fifty patient applications (each with a different patient/user), and two third party applications, the single healthcare provider application comprises fifty interfaces, one interface corresponding to each patient/user using a patient application on the platform. Similarly, for example, in an embodiment of a platform including a plurality of healthcare provider applications (each corresponding to a different healthcare provider), a patient application may comprise two or more interfaces, wherein a single interface corresponds to a different healthcare provider using a healthcare provider application on the platform.

In general, various embodiments of interfaces as described herein comprise different components that are configured to present or receive input in different data formats including, for example, graphical formats, audio formats, and video formats.

In some embodiments, an interface comprises one or more portals configured to provide interactive data that, in some embodiments, is customized to a type of user.

In general, a healthcare provider interface in a healthcare provider application is configured to: (1) simplify the complexities involved in providing patient care and (2) integrate patient input into the healthcare provider decision making process. In general, addressing the complexities involved in providing patient care is achieved by providing the data insights engine output to a healthcare provider within the healthcare provider interface. In general, integrating patient input in the decision making process is achieved by providing input provided by a patient within the healthcare provider interface as well as, in some embodiments, providing the patient input to the data insights engine which may result in a particular output from the data insights engine that is based at least in part on the patient input.

Data is typically organized, presented, and/or received in a healthcare provider application, patient application, and/or a third party application within a portal (or a portion of a portal). A portal typically comprises interactive links, images, and/or buttons that allow a user to directly interact with the content that is displayed in the portal. Non-limiting examples of content displayed in a portal include healthcare provider identifying data, patient identifying data, third party identifying data, patient health data, and output(s) from a data insights engine. In some embodiments, a portal includes a plurality of screens that include different content from one another. In some embodiments, a healthcare provider has a single portal in which he or she is able to access the health data of all of his or her patients. In some embodiments, a healthcare provider has a single portal for each one of his or her patients (i.e., each patient has an individual portal). Likewise, in some embodiments, if a patient is cared for by more than one healthcare provider, he or she has an individual portal for each healthcare provider. Likewise, in some embodiments, a healthcare provider has an individual portal for each third party that he or she interacts with and, in some embodiments, a healthcare provider portal is configured to be used for third party interactions in addition to other functions including patient related functions.

In some embodiments, one or more portals of a custom interface (e.g., healthcare provider interface, patient interface, and/or third party interface) are customized, for example to a specific type of use and/or specific type of data.

In some embodiments, one or more portals of a patient interface of a patient application enable the patient to, for example, obtain physiological data from a sensing device and/or from other health-based software applications, store sensed data from a sensing device, transmit and receive communications, track completion of tasks, and/or transmit physiological, audio, and visual data to a healthcare provider application. In some embodiments of the patient application, the patient application is configured to communicate with another application running within the platform described herein, and, in some embodiments, is also configured to communicate with software applications that are not a part of the platform. In some embodiments, data transmitted to a healthcare provider application from the patient application comprises, for example, height data, weight data, age data, physical activity level data, heart rate data, blood pressure data, and ECG data.

In some embodiments, a patient application is configured to receive a communication from a patient in one or more of written format, audio format, and/or video format. Said communication is inputted into the patient application and the patient is then able to transmit the communication to a healthcare provider application.

In some embodiments, a patient interface is configured to allow a patient to provide feedback (or input) regarding their treatment plan, treatment regimen, and/or treatment goals. Non-limiting examples of patient feedback (or input) include data regarding symptoms, data regarding side-effects of a treatment, and a change to a previously set goal. For example, a patient may wish to change a treatment goal in view of an upcoming important event. That is, a goal may be to, for example, not have side effects during a marriage of a child scheduled on a specific date. A healthcare provider application in these embodiments, is configured to receive the patient feedback (or input) and where needed adjust accordingly. In the example, of the goal change for the wedding of a family member, a treatment regimen may be, for example, paused or modified in the period leading up to the planned wedding in order to reduce or eliminate the risk of side effects occurring to the patient during the wedding.

A healthcare provider interface, in some embodiments, is configured to receive and display patient data, communications, and feedback (or input). Non-limiting examples of patient data are height, weight, body mass index (BMI), age, physical activity level, heart rate, blood pressure, temperature, and/or ECG data. Non-limiting examples of patient communications include written, audio, and video recorded communications. Non-limiting examples of feedback include feedback (or input) regarding symptoms, side effects, and goals.

The healthcare provider interface may alert the healthcare provider if certain data, communications, and/or feedback is received. For example, an interface of the healthcare provider's software interface may show a list of notifications displaying data such as the patient's communication. In some embodiments, a notification may indicate the receipt of feedback from the patient. In some embodiments, a healthcare provider is able to configure the healthcare provider interface to provide a notification when certain data, communication, and/or feedback is received. For example, if a patient provides feedback that they are experiencing chest pain, a notification may be configured to sound an auditory alarm once the communication is received by the healthcare provider interface. A healthcare provider interface, in some embodiments, may also be configured to issue automated responses to received data, communication, and/or feedback from a patient. For example, an automated response may solicit more data from the patient, may direct the patient to carry out an activity, or may congratulate the patient for achieving a certain task or goal indicated by the received data, communication, and/or feedback. Automated communications to a patient, in some embodiments, may also be configured to be regularly sent (including not in response to any received communication). In these embodiments, an automated communication from a healthcare provider to a patient may be configured to solicit feedback from the patient regarding the treatment regimen that they are receiving.

In some embodiments, a healthcare provider's interface may comprise a viewable, interactive patient directory and/or database. The patient directory and/or database may comprise a list of all patients receiving care from the healthcare provider. The patient directory may further display patient data such as name, phone number, age, gender, and an indication of whether or not they are using the patient interface. Such interface component may be placed in close proximity to the patient's name (or patient image) for ease of accessibility. The healthcare provider interface of the healthcare provider interface may also comprise a component to provide the healthcare provider with the option to add a new patient.

In some embodiments, a third party interface is a part of the platform, wherein non-limiting examples of third parties with a third party interface include healthcare payers, pharmaceutical developers, medical device developers, research institutions, and hospitals. In some embodiments, a third party interface receives health data relating to patients receiving treatment. In some embodiments, a third party interface receives an output from a data insights engine. Said output may, for example, relate to an effectiveness of a treatment regimen or component thereof. The data provided to a third party interface, in some embodiments, is determined by a healthcare provider in the healthcare provider interface.

In general, each interface described herein is configured to communicate with a data insights engine. In some embodiments, each portal within an interface is configured to receive data from or transmit data to (or both) to a data insights engine as described herein. In some embodiments, data to be transmitted to or received from (or both) a data insights engine is/are directly via portal without being initiated by a user, whereas, in some embodiments, data transmission or data receipt (or both) from a data insights engine must be initiated by an action of a user, such as, for example, activating a hyperlink or voice command.

### Digital Processing Device

In some embodiments, the platforms and methods described herein include a digital processing device or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft' Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, and/or stylus. In some embodiments, the input device is a touch screen or a multi-touch screen. In other embodiments, the input device is a microphone to capture voice or other sound input. In other embodiments, the input device is a video camera or other sensor to capture motion or visual input. In still further embodiments, the input device is a combination of devices such as those disclosed herein.

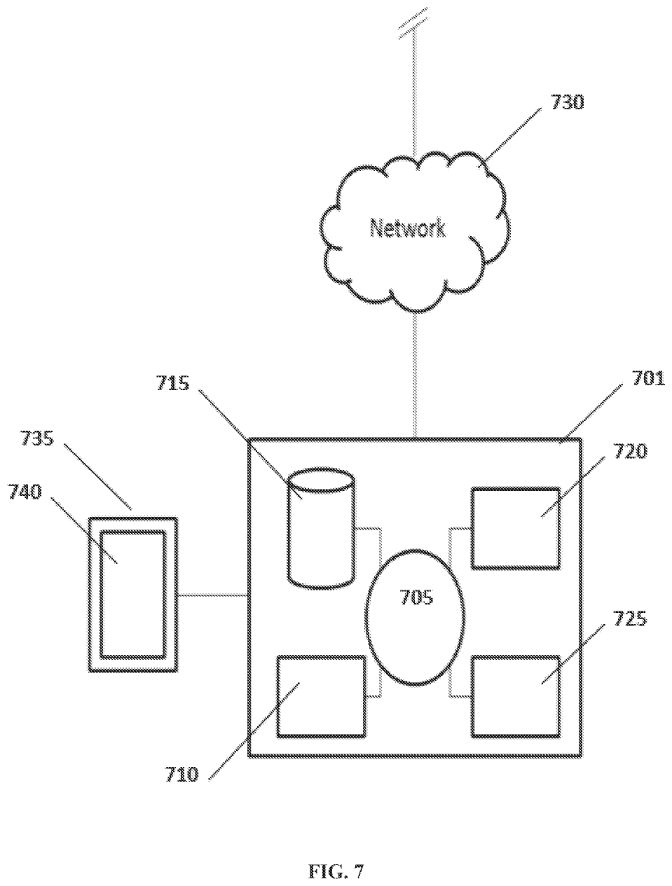

FIG. 7 shows an exemplary digital processing device 701 programmed or otherwise configured to store profiles, ingest health data from external sources, value individual profiles, and/or provide interfaces for searching profiles. In this embodiment, the digital processing device 701 includes a central processing unit (CPU, also “processor” and “computer processor” herein) 705, which can be a single core or multi core processor, or a plurality of processors for parallel processing. The digital processing device 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The digital processing device 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 1530 can include one or more computer servers, which can enable distributed computing, such as cloud computing. The network 730, in some cases with the aid of the device 701, can implement a peer-to-peer network, which may enable devices coupled to the device 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and write back. The CPU 705 can be part of a circuit, such as an integrated circuit. One or more other components of the device 701 can be included in the circuit. In some cases, the circuit is an application specific integrated circuit (ASIC) or a field programmable gate array (FPGA).

The storage unit 715 can store files, such as drivers, libraries and saved programs. The storage unit 715 can store user data, e.g., user preferences and user programs. The digital processing device 701 in some cases can include one or more additional data storage units that are external, such as located on a remote server that is in communication through an intranet or the Internet.

The digital processing device 701 can communicate with one or more remote computer systems through the network 730. For instance, the device 701 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

### Non-transitory Computer Readable Storage Medium

In some embodiments, the platforms and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle® Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

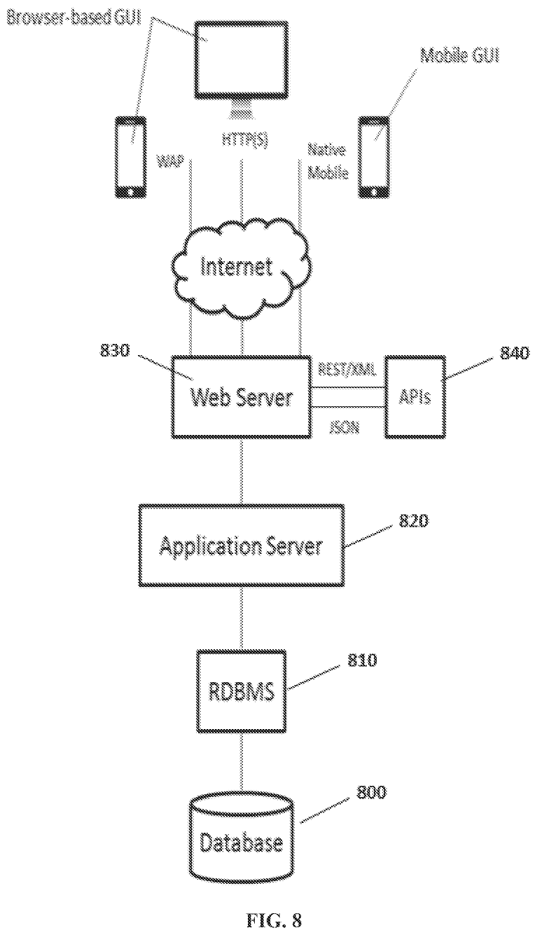

FIG. 8 shows an exemplary application provision system which comprises one or more databases 800 accessed by a relational database management system (RDBMS) 810. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 820 (such as Java servers, .NET servers, PHP servers, and the like) and one or more web servers 830 (such as Apache, IIS, GWS and the like). The web server(s) optionally expose one or more web services via app application programming interfaces (APIs) 840. Via a network, such as the Internet, the system provides browser-based and/or mobile native user interfaces.

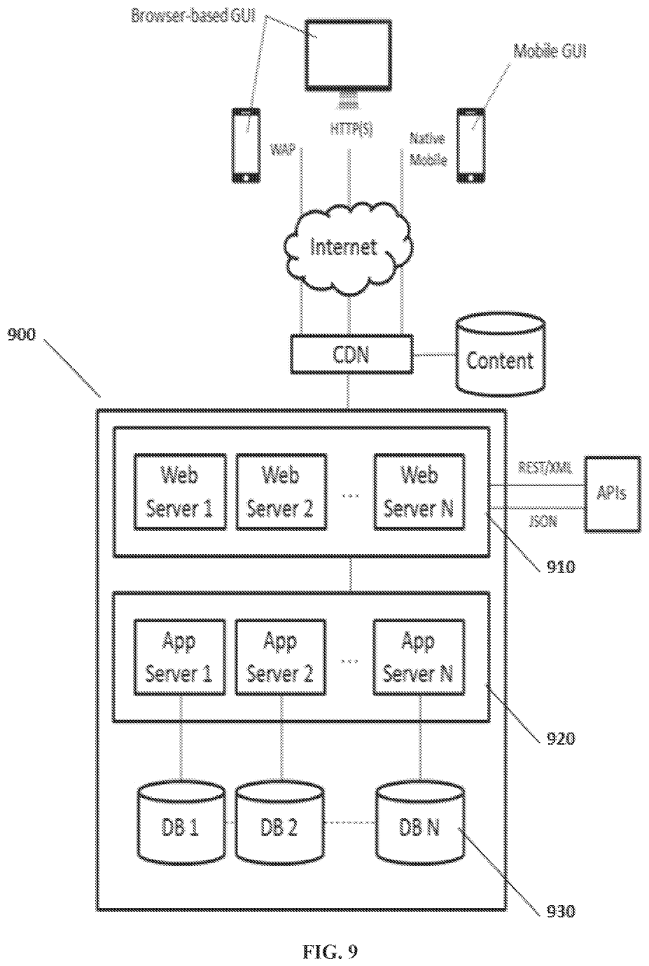

FIG. 9 shows an exemplary application provision system which alternatively has a distributed, cloud-based architecture 900 and comprises elastically load balanced, auto-scaling web server resources 910 and application server resources 920 as well synchronously replicated databases 930.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®^{,} Mozilla® Firefox®, Google® Chrome, Apple® Safari®, Opera Software' Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of profile, fitness, genetic, health, profile value, and trust information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A computer-implemented system for data state alignment and cohort generation, the system comprising:
a processor;
a non-transitory computer readable storage medium encoded with a computer program that causes said processor to:
receive user input for querying a database comprising a plurality of reference data states based on one or more subject data states;
align said one or more subject data states with said plurality of reference data states;
identify one or more groups of similar reference data states based on aligning said one or more subject data states with said plurality of reference data states; and
generate summary statistics comprising one or more analytical insights based on said one or more groups of similar reference data states.

2. (canceled)

3. The system of claim 1, wherein said processor is further caused to extract one or more intents from said user input, wherein said one or more intents define at least one target variable, exclusion criteria, a type of comparison, or any combination thereof.

4. (canceled)

5. (canceled)

6. The system of claim 3, wherein said type of comparison comprises an individualized comparison, a group comparison, or a ranking.

7. (canceled)

8. (canceled)

9. (canceled)

10. (canceled)

11. (canceled)

12. (canceled)

13. (canceled)

14. The system of claim 1, wherein said plurality of reference data states corresponds to multiple time points for a plurality of reference subjects, wherein each of said plurality of reference data states corresponds to a time point for a reference subject.

15. (canceled)

16. (canceled)

17. (canceled)

18. The system of claim 14, wherein said one or more groups of similar reference data states are identified using the most similar reference data state for each of said plurality of reference subjects.

19. The system of claim 1, wherein said one or more subject data states comprises subject information, and wherein said subject information comprises a plurality of features comprising one or more of: (U age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, (15) treatment goal, (16) socioeconomic factors, or (17) confounding factors.

20. (canceled)

21. The system of claim 19, wherein said processor is further configured to parse said one or more subject data states into said one or more groups based on said subject information.

22. The system of claim 1, wherein said processor is further configured to recommend and answer one or more questions determined to be associated with said user input.

23. (canceled)

24. The system of claim 1, wherein said processor is further caused to output said summary statistics comprising said one or more analytical insights.

25. The system of claim 24, wherein said processor is further caused to generate and output a comparison of a group comprising said one or more subject data states to another group, population, or a reference material, and wherein said comparison compares summary statistics for said group with said another group, said population, or said reference material.

26. (canceled)

27. The system of claim 1, wherein said one or more groups of similar reference data states is determined to be most similar to said one or more subject data states, and wherein either: (i) said processor is further caused to output feature importance for one or more features used to determine said one or more groups of similar reference data states as being most similar to said one or more subject data states or (ii) said processor is further caused to output a ranked list of reference data states selected from said plurality of reference data states most similar to said one or more subject data states.

28. (canceled)

29. (canceled)

30. The system of claim 1, wherein said one or more groups of similar reference data states are identified using a similarity algorithm configured to determine a statistical similarity between said one or more subject data states and said plurality of reference data states, and wherein said similarity algorithm is a non-machine learning algorithm.

31. (canceled)

32. (canceled)

33. The system of claim 1, wherein said one or more subject data states comprises health data for one or more subjects retrieved from an electronic medical record, and wherein said health data comprises a plurality of features extracted from said electronic medical record or other health data sources using a natural language processing algorithm, and wherein said natural language processing algorithm comprises one or more rules for keyword identification, unit conversion, internal consistency, named entity recognition, relationship extraction, or other natural language processing methods or any combination thereof.

34. (canceled)

35. (canceled)

36. The system of claim 33, wherein said natural language processing algorithm comprises a natural language processing model configured to annotate said electronic medical record with gold standard labels, wherein said processor is further configured to provide a healthcare provider application comprising a healthcare provider interface configured to present a healthcare provider with said statistical summary comprising said one or more analytical insights.

37. (canceled)

38. The system of claim 36, wherein said healthcare provider interface comprises a first plurality of portals, wherein at least one of said first plurality of portals comprises a patient context data grouping comprising a subset of said reference data states within said one or more groups.

39. The system of claim 38, wherein at least one of said first plurality of portals comprises an outcomes navigator data grouping comprising outcome information for said subset of said reference data states within said one or more groups, and wherein said outcome information comprises one or more of cancer survival, cancer progression, adverse event status, treatment status, acute care, or mortality.

40. (canceled)

41. (canceled)

42. (canceled)

43. A computer-implemented method for data state alignment and cohort generation, comprising:
receiving user input for querying a database comprising a plurality of reference data states based on one or more subject data states;
aligning said one or more subject data states with said plurality of reference data states;
identifying one or more groups of similar reference data states based on aligning said one or more subject data states with said plurality of reference data states; and
generating summary statistics comprising one or more analytical insights based on said one or more groups of similar reference data states.

44. (canceled)

45. The method of claim 43, further comprising extracting one or more intents from said user input, wherein said one or more intents define at least one target variable, exclusion criteria, a type of comparison, or any combination thereof, and wherein said type of comparison comprises an individualized comparison, a group comparison, or a ranking.

46. (canceled)

47. (canceled)

48. (canceled)

49. (canceled)

50. (canceled)

51. (canceled)

52. (canceled)

53. (canceled)

54. (canceled)

55. (canceled)

56. The method of claim 43, wherein said plurality of reference data states corresponds to multiple time points for a plurality of reference subjects, and wherein each of said plurality of reference data states corresponds to a time point for a reference subject.

57. (canceled)

58. (canceled)

59. (canceled)

60. The method of claim 56, wherein said one or more groups of similar reference data states are identified using the most similar reference data state for each of said plurality of reference subjects.

61. The method of claim 43, wherein said one or more subject data states comprises subject information, wherein said subject information comprises a plurality of features comprising one or more of: (1) age, (2) gender, (3) race, (4) exposure, (5) co-morbidity, (6) diagnosis, (7) prognosis, (8) tumor pathology, (9) serum markers, (10) radiology findings, (11) family history, (12) surgical history, (13) treatment plan, (14) treatment regimen, or (15) treatment goal.

62. (canceled)

63. The method of claim 61, further comprising parsing said one or more subject data states into said one or more groups based on said subject information.

64. (canceled)

65. (canceled)

66. The method of claim 43, further comprising outputting said summary statistics comprising said one or more analytical insights.

67. The method of claim 66, further comprising generating and outputting a comparison of a group comprising said one or more subject data states to another group, population, or a reference material.

68. (canceled)

69. The method of claim 43, wherein said one or more groups of similar reference data states is determined to be most similar to said one or more subject data states.

70. The method of claim 69, further comprising outputting feature importance for one or more features used to determine said one or more groups of similar reference data states as being most similar to said one or more subject data states.

71. The method of claim 69, further comprising outputting a ranked list of reference data states selected from said plurality of reference data states most similar to said one or more subject data states.

72. The method of claim 43, wherein said one or more groups are identified using a similarity algorithm configured to determine statistical similarity between said one or more subject data states and said plurality of reference data states, wherein said similarity algorithm is a non-machine learning algorithm.

73. (canceled)

74. (canceled)

75. The method of claim 43, wherein said one or more subject data states comprises health data for one or more subjects retrieved from an electronic medical record, and wherein said health data comprises a plurality of features extracted from said electronic medical record or other health data sources using a natural language processing algorithm, and wherein said natural language processing algorithm comprises one or more rules for keyword identification, unit conversion, internal consistency, named entity recognition, relationship extraction, or other natural language processing methods, or any combination thereof.

76. (canceled)

77. (canceled)

78. The method of claim 75, wherein said natural language processing algorithm comprises a natural language processing model configured to annotate said electronic medical record with gold standard labels.

79. The method of claim 78, further comprising providing a healthcare provider application comprising a healthcare provider interface configured to present a healthcare provider with said statistical summary comprising said one or more analytical insights, wherein said healthcare provider interface comprises a first plurality of portals, wherein at least one of said first plurality of portals comprises a patient context data grouping comprising a subset of said reference data states within said one or more groups.

80. (canceled)

81. The method of claim 79, wherein at least one of said first plurality of portals comprises an outcomes navigator data grouping comprising outcome information for said subset of said reference data states within said one or more groups and wherein said outcome information comprises one or more of cancer survival, cancer progression, adverse event status, treatment status, acute care, or mortality.

82. (canceled)

83. (canceled)

84. (canceled)
